(19) United States
(12) Patent Application Publication
(10) Pub. No.: US 2023/0094765 A1
(43) Pub. Date: Mar. 30, 2023

(54) AUTOMATED SAMPLE EXTRACTION APPARATUS AND METHOD

(71) Applicant: John Richard Nobile, Guilford, CT (US)

(72) Inventor: John Richard Nobile, Guilford, CT (US)

(21) Appl. No.: 17/935,423

(22) Filed: Sep. 26, 2022

Related U.S. Application Data
(60) Provisional application No. 63/248,533, filed on Sep. 26, 2021.

(51) Int. Cl. B01L 3/00

(52) U.S. Cl. CPC B01L 3/502753; B01L 3/505; B01L 3/502738; C12Q 1/6806

(57) ABSTRACT

An automatic nucleic acid extraction cartridge and an automatic nucleic acid extraction system including the same are described herein. The cartridge having a housing that includes a sample port, a cell processing chamber, a wash fluid chamber, a filter assembly comprising a filter member, and a diverter valve having a first and a second reversibly sealable output, wherein each of the sample port and the cell processing chamber, the cell processing chamber and the filter assembly, and the wash fluid chamber and the filter assembly are in one-way fluid communication, and the filter assembly is in fluid communication with the diverter valve and (i) a waste conduit when the diverter valve is biased to the first reversibly sealable output and (ii) a pathogen nucleic acid conduit when the diverter valve is biased to the second reversibly sealable output. The present disclosure further describes methods of using the same.

### CROSS-REFERENCE TO RELATED APPLICATIONS

The present disclosure claims priority to and the benefit of U.S. Provisional Patent Application Ser. No. 63/248,533, filed 26 Sep. 2021, which is incorporated by reference herein in its entirety for all purposes.

### FIELD OF THE INVENTION

The invention of the present disclosure provides an automatic nucleic acid extraction cartridge and an automated nucleic acid extraction instrument, each of which can be parts of the system, and facilitate the quick, automated method of extracting nucleic acids from one or more pathogens from a liquid sample, such as a liquid sample obtained from a subject. The present disclosure further provides a mixing apparatus and a diverter valve that may be used in the cartridges, instruments, and/or systems of the present disclosure, as well as a method of quickly extracting nucleic acids of one or more pathogens from a liquid sample, such as a liquid sample obtained from a subject, and associated methods.

### BACKGROUND

Present techniques for nucleic acid extraction require expensive equipment and technicians with significant training. As result, a need exists for a system and methods to extract nucleic acids in resource poor environments. The present disclosure

### SUMMARY OF THE INVENTION

The present disclosure describes an automatic nucleic acid extraction cartridge, unit, or system, as well as an automated nucleic acid extraction system comprising the cartridge of the present disclosure. The present disclosure further describes a novel and inventive mixing apparatus and diverter valve that can be utilized in the automatic nucleic acid extraction cartridge described herein and the automated nucleic acid extraction system described herein. The apparatuses, systems, and methods described herein provide a way to quickly and easily extract nucleic acids, such as nucleic acids from one or more pathogens, from a liquid biological sample.

An aspect of the present disclosure relates to an automatic nucleic acid extraction cartridge or unit. The cartridge, unit, or system comprises a housing including (a) a sample port that receives a liquid biological sample; (b) a cell processing chamber (for example, a cell lysis chamber); (c) a wash fluid chamber; (d) a filter assembly comprising a filter member; and (e) a diverter valve (for example, a zero dead volume diverter valve) having a first reversibly sealable (or closable) output and a second reversibly sealable (or closable) output, wherein (1) the sample port is in one-way fluid communication with the cell processing chamber, (2) the cell processing chamber is in one-way fluid communication with the filter assembly, (3) the wash fluid chamber is in fluid communication (e.g., one-way fluid communication) with the filter assembly, and (4) the filter assembly is in fluid communication (e.g., one-way fluid communication) with the diverter valve and (i) a waste conduit when the diverter valve is biased to the first reversibly sealable (or closable) output and (ii) a pathogen nucleic acid conduit when the diverter valve is biased to the second reversibly sealable (or closable) output.

In any aspect or embodiment described herein, one or more of the following: (a) the liquid sample container comprises a liquid sample contained therein; (b) the cell processing chamber comprises a processing solution contained therein; (c) the cell processing chamber comprises a mixing apparatus contained therein; (d) the wash fluid chamber comprises a wash solution (for example, a wash buffer, such as phosphate buffered saline (PBS) or water)) contained therein; (e) the filter member is configured to retain one or more pathogens; (f) the one or more pathogens comprises one or more bacteria, one or more virus, one or more eukaryotic pathogen, or a combination thereof; (g) the first reversibly sealable (or closable) output includes a region that can be reversibly pinched, compressed, or crimped to be in a sealed (or closed) position that does not allow fluid to pass through; (h) the second reversibly sealable (or closable) output includes a region that can be reversibly pinched, compressed, or crimped to be in a sealed (or closed) position that does not allow fluid to pass through; (i) the first reversibly sealable (or closable) output and the second reversibly sealable (or closable) output are both in a sealed (or closed) position; (j) the sample port and the cell processing chamber, the cell processing chamber and the filter assembly, the wash fluid chamber and the filter assembly, the filter member or filter assembly and the diverter valve are connected via a conduit; or (k) a combination thereof.

In any aspect or embodiment described herein, the cartridge further comprises: (a) a sampling device that is inserted into a liquid sample container (for example, the sampling device punctures the liquid sample container, such as when the liquid sample container is being placed into a liquid sample container holder); (b) a liquid sample container holder; (c) a first pressure exerting device in fluid communication with the cell processing chamber (or lysing chamber); (d) a second pressure exerting device in fluid communication with the wash fluid chamber; (e) an extracted nucleic acid receptacle that is in fluid communication with the pathogen nucleic acid conduit; (f) a waste reservoir that is in fluid communication with the waste conduit or the first reversibly sealable (or closable) output; or (g) a combination thereof.

In any aspect or embodiment described herein, the filter assembly further comprises: (a) a first thermally conductive element (for example, a thermally conductive metal element) that transmits thermal energy to the filter; (b) a metal element that transmits at least vibrational energy (for example, sound waves, sonic energy, ultrasonic energy, etc.) to the filter member (for example, the metal element transmits both vibrational energy and thermal energy to the filter member); or (c) a combination thereof.

In any aspect or embodiment described herein, one or more of the following: (a) the conduit connecting of the sample port and the cell processing chamber comprises a first one-way valve (for example, a pin valve); (b) the conduit of the cell processing chamber and the filter assembly comprises a second one-way valve (for example, a pin valve or diaphragm valve); (c) the conduit of the wash fluid chamber and the filter assembly comprises a third one-way valve (for example, a pin valve or a plug valve); or (d) a combination thereof.

In any aspect or embodiment described herein, the first one-way valve, the second one-way valve, the third one-way valve, or a combination thereof, comprise (i) a conduit having a hollow interior that includes a straight interior region and an expanding conical interior region located at the end of the conduit that fluid exits the one-way valve (for example, the expanding conical interior region or the entire conduit can be made with an elastomeric or plastomeric material), and (ii) a partially conical elastomer valve pin or a frustoconical elastomeric valve pin (for example, a rubber pin) that mates with the expanding conical interior region of the conduit, thereby causing a substantially or completely fluid tight (for example, liquid-tight or water-tight seal) to form when there is flow or positive pressure from a fluid contacting the larger end of the valve pin.

In any aspect or embodiment described herein, one or more of the following: (a) the valve pin comprises an extended cylindrical area that extends from the small end of the pin and that is smaller in diameter from the straight interior region of the one-way valve; (b) the taper angle of the expanding conical interior region and the valve pin is about 12 degrees to about 24 degrees; (c) the valve pin has a slightly greater taper angle than the expanding conical interior region of the valve (e.g., the valve pin has a taper angle that is about 0.5 to about 1 degree greater than the expanding conical interior region of the valve); or (d) a combination thereof.

In any aspect or embodiment described herein, one or more of the following: (a) the filter member has a pore size that retains one or more pathogens (for example, a pore size of about 0.45 μm or less—such as, about 0.45 μm, about 0.4 μm, about 0.3 μm, about 0.22 μm, about 0.2 μm, or about 0.1 μm); (b) the filter member has a coating (e.g., a composition, a ligand, a peptide, an antibody, etc.) that has an affinity for or captures one or more pathogens; (c) the filter member is statically charged; (d) the filter member is made of a material that does not induce a fibrinogen-driven clotting reaction (for example, the filter member is a polycarbonate filter or a polyester filter); or (e) a combination thereof.

In any aspect or embodiment described herein, one or more of the following: (a) the sampling device is inserted into the liquid sample in an upward direction; (b) the sampling device punctures the liquid sample container (e.g., the liquid sample container includes a septum that the sampling device punctures and/or the sampling device punctures the septum in an upward direction); (c) the sampling device includes a sampling needle and a venting needle, each being inserted into the liquid sample in the liquid sample container (for example, the sampling device punctures the liquid sample container when being placed into the holder and/or inserted into the liquid sample); or (d) a combination thereof.

In any aspect or embodiment described herein, one or more of the following: (a) the sampling needle has (i) a larger transfer capacity than the venting needle (for example, a larger diameter when the needle has a circular cross-section or a larger side when the needle has a square or rectangular cross-section), (ii) a shorter length than the venting needle (i.e., the end of the sampling needle is located lower in the liquid sample container than the end of the venting needle, preferably the sampling needle is located close to the bottom of the liquid sample container, the venting needle is located close to the top of the liquid sample container, or both), or (iii) both; (b) the venting needle vents to the waste reservoir and/or the atmosphere, or (c) a combination thereof.

In any aspect or embodiment described herein, the mixing apparatus is a rotary mixer comprising a mixing body (for example, a plastic mixing body).

In any aspect or embodiment described herein, one or more of the following: (a) the cell processing chamber includes a circular cross-section (e.g., located at the bottom of the cell processing chamber) and the rotary mixer comprises a cylindrical mixing body that rotates freely within the circular cross-section of the cell processing chamber; (b) the rotary mixer further comprises two or more (e.g., 2, 3, 4, 5, 6, 7, 8, 9, or 10, preferably 4) magnets inserted into (for example, located internally) the mixing body (for example, the rotary mixer with two or more magnets that can interact with a rotating external magnetic field, thereby causing rotation of the rotary mixer); or (c) a combination thereof.

In any aspect or embodiment described herein, one or more of the following: (a) the two or more magnets are substantially evenly spaced or substantially symmetrically located about the rotational axis of the rotary mixer; (b) the two or more magnets have the same orientation of their polarity (i.e., the north pole of each magnet is facing outward or inward in the mixing apparatus or rotary mixer); (c) the mixing apparatus further comprises fluid engagement features extending upward from the cylindrical mixing body; (d) the cylindrical mixing body further comprises an open center, or (e) a combination thereof.

In any aspect or embodiment described herein, one or more of the following: (i) the fluid engagement features are smooth enough to minimize cavitation or substantially eliminate cavitation, (ii) the fluid engagement features have, or the top of the cylindrical mixing body has, a substantially sinusoidal shape (for example, sinusoidal shape), (iii) the fluid engagement features create a vortex when spinning about a vertical axis, or (iv) a combination thereof.

In any aspect or embodiment described herein, one or more of the following: (a) the first pressure exerting device is capable of providing positive pressure and negative pressure to the cell processing chamber; (b) the first pressure exerting device moves fluid (for example, liquid sample) into the cell processing chamber when a negative pressure is applied by the first pressure exerting device to the cell processing chamber; (c) the first pressure exerting device moves fluid (for example, processed liquid sample) into the filter assembly when a positive pressure is applied by the first pressure exerting device to the cell processing chamber; (d) the second pressure exerting device is capable of providing at least positive pressure to the wash fluid chamber; (e) the filter member or assembly retains one or more pathogens when the second pressure exerting device applies a positive pressure to the wash fluid chamber, thereby passing a portion of the wash solution through the filter assembly; (f) the extracted nucleic acid receptacle (e.g., a microfuge tube) receives the extracted nucleic acids when the second pressure exerting device passes a portion of the wash solution through the filter assembly after the one or more pathogens are lysed from the second reversibly sealable (or closable) output; or (g) a combination thereof.

In any aspect or embodiment described herein, the automated nucleic acid extraction cartridge further comprises a first syringe holder that accepts a first syringe, wherein the cell processing chamber is the hollow cylinder of the first syringe and the first pressure exerting device is a sliding plunger of the first syringe (for example, the syringe holder receives the first syringe between the first one-way valve and the second one-way valve—upstream of the first one-way valve and downstream of the second one-way valve).

In any aspect or embodiment described herein, the first pressure exerting device is a processing port that is capable of engaging a pressure driver from an external instrument (e.g., the first driver of the automated nucleic extraction system described herein) that provides positive pressure and negative pressure to the processing port.

In any aspect or embodiment described herein, the processing port seals (for example, a hermetic seal) the cell processing chamber (for example, seals the cell processing chamber from the external environment).

In any aspect or embodiment described herein, the automated nucleic acid extraction cartridge further comprises a second syringe holder that accepts a second syringe, wherein the wash fluid chamber is a hollow cylinder of the second syringe and the second pressure exerting device is a sliding plunger of the second syringe.

In any aspect or embodiment described herein, the second pressure exerting device is a wash fluid chamber port that is capable of engaging a pressure driver from an external instrument (e.g., the second driver of the automated nucleic extraction system described herein) that provides at least positive pressure to the wash fluid chamber port.

In any aspect or embodiment described herein, the wash fluid chamber port seals (for example, a hermetic seal) the wash fluid chamber from the external environment.

In any aspect or embodiment described herein, one or more of the following: (a) the processing solution makes the non-pathogenic components of the liquid sample filterable (for example, filterable without lysing eukaryotic cells, filterable by lysing non-pathogenic eukaryotic cells, etc.) without lysing one or more bacterial pathogens, one or more eukaryotic pathogens, one or more viral pathogens, or a combination thereof; (b) the processing solution is a hypertonic solution relative to one or more non-pathogen eukaryotic cells of the liquid sample (for example, one or more eukaryotic cells of a subject from which the liquid sample was obtained from); or (c) the processing solution (i) does not lyse or break down one or more prokaryotic pathogens, (ii) does not lyse or breakdown one or more eukaryotic pathogens, (iii) does not lyse or break down one or more viral pathogens, (iv) lyses blood cells (for example, red and/or white blood cells), (v) breaks down proteins, (vi) breaks down nucleic acids, (vii) suppresses clotting, (viii) lyses non-pathogen eukaryotic cells, or (ix) a combination thereof; or (d) a combination thereof.

A further aspect of the present disclosure is an automated nucleic acid extraction system comprising the automated nucleic acid extraction cartridge of the present disclosure and a system body that comprises: (a) a first driver that provides the force to the first pressure exerting device to exert the negative pressure to the cell processing chamber and the positive pressure to the cell processing chamber; (b) a second driver that provides the force to the second pressure exerting device to exert a positive pressure to the wash fluid chamber; (c) an external magnetic field (ExMF) creating device that creates an ExMF that drives the mixing apparatus; (d) a pathogen lysing device that engages the filter assembly (for example, the filter member) and that lyses the one or more pathogens contained in the filter assembly (for example, on the filter member); and (e) a diverter valve control unit that controls the diverter valve.

In any aspect or embodiment described herein, one or more of the following: (a) the system body further comprising a control unit that controls the first driver, the second driver, the ExMF creating device, the pathogen lysing device, the diverter valve, or a combination thereof; (b) the system further comprising a system door that is attached to and articulated with the system body between an open position in which the cartridge is accessible and a closed position that produces an enclosed space where the cartridge is placed or located; or (c) a combination thereof.

In any aspect or embodiment described herein, one or more of the following: (a) the mixing apparatus is a rotary mixer; and (b) the ExMF creating device includes (i) two or more (e.g., 2, 3, 4, 5, 6, 7, 8, 9, or 10, preferably 6) field or stator coils that are sequentially energized and that are placed around the cell processing chamber (for example, the hollow cylinder of the first syringe) and in the same field as the rotatory mixer; or (ii) two or more synchronized magnets that are rotated around the cell processing chamber (for example, the hollow cylinder of the first syringe) and in the same plane as the rotary mixer.

In any aspect or embodiment described herein, the ExMF creating device includes two arms that together surround the cell processing chamber (for example, the hollow cylinder of the first syringe) for creating the ExMF, each arm including one or more (e.g., 1, 2, 3, or 4, preferably 3) substantially evenly spaced or substantially symmetrically located field or stator coils.

In any aspect or embodiment described herein, the two arms are articulated (e.g., articulated laterally) between a closed position that places the two arms around the cell processing chamber (for example, the hollow cylinder of the first syringe) for creating the ExMF and an open position that permits the cell processing chamber to be positioned between the two arms.

In any aspect or embodiment described herein, one of the two arms of the ExMF creating device is mounted on the system body and the second of the two arms of the ExMF creating device is mounted on the system door.

In any aspect or embodiment described herein, the ExMF creating device: (a) senses the position of the one or more magnets of the rotary mixer relative to the field or stator coil using the back electromagnetic field (EMF) created by the permanent magnets of the rotary mixer passing by the field or stator coils; (b) continuously examines whether the rotary mixer is rotating (e.g., rotating properly or as intended); (c) provides continuous intimation of how the rotary mixer is rotating; or (d) a combination thereof.

In any aspect or embodiment described herein, the mixing apparatus (i) mixes at about 500 to about 7000 rotations per minute (RPM) (for example, about 500 to about 7000 RPM, wherein the speed is increased during the mixing process, about 500 to about 3000 RPM, about 2,000 to about 7000 RPM, or about 4000 to 7000 RPM), (ii) mixes at least while the liquid sample is introduced into the cell processing chamber, (iii) mixes the liquid sample and the processing solution for about 3 to about 10 minutes, or (iv) a combination thereof.

In any aspect or embodiment described herein, the diverter valve control unit comprises an actuator or pinching member that has (i) a first position that pinches, compresses, or crimps (that is, a sealed or closed position) for the first reversibly sealable (or closable) output, (ii) a second position that pinches, compresses, or crimps (that is, a sealed or closed position) for the second reversibly sealable (or closable) output, and (iii) a third position that does not pinch, compress, or crimp either of the first or the second reversibly sealable (or closeable) outputs to the point of stopping the flow of fluid (that is, an unsealed or opened position).

In any aspect or embodiment described herein, the diverter valve control unit comprises a first actuator or pinching member for the first reversibly sealable (or closable) output and a second actuator or pinching member for the second reversibly sealable (or closable) output, wherein each actuator or pinching member has a first position that seals (or closes) the output and a second position that opens the output.

In any aspect or embodiment described herein, the first position of each of the actuator or pinching member closes the output by pinching, compressing, or crimping a region of the output that can be reversibly pinched, compressed, or crimped.

In any aspect or embodiment described herein, the pathogen lysing device comprises (a) a heater that heats contents of the filter assembly to a temperature sufficient to lyse the one or more pathogens (for example, at least 100° C.); (b) a sonic or ultrasonic wave transmitter (for example, sound waves of 20,000 kHz or greater) that transmits sound waves (for example, sonic or ultrasonic energy) to contents of the filter assembly (for example, for about 15 seconds to about 5 minutes) that are sufficient to lyse the one or more pathogens; or (c) a combination thereof.

In any aspect or embodiment described herein, one or more of the following: (a) the first driver further comprises at least one force sensor that detects how much force is being exerted on the cell processing chamber, (b) the second driver further comprises at least one force sensor that detects how much force is being exerted on the wash fluid chamber, or (c) a combination thereof.

In any aspect or embodiment described herein, the system body further comprises a waste reservoir that is in fluid communication with (that is, receives fluid from) the end of the waste conduit not connected to the diverter valve (for example, the automated nucleic acid extraction system does not contain a waste reservoir).

An additional aspect of the present disclosure relates to a mixing apparatus (13) that comprises a cylindrical mixing body that comprises two or more (e.g., 2, 3, 4, 5, 6, 7, 8, 9, or 10, preferably 4) magnets inserted into (for example, located internally) the cylindrical mixing body.

In any aspect or embodiment described herein, one or more of the following: (i) the two or more magnets have the same orientation of their polarity (i.e., the north pole of each magnet is facing outward or inward in the mixing apparatus or rotary mixer), (ii) the mixing apparatus (13) further comprises fluid engagement features extending upward from the cylindrical mixing body, (iii) the cylindrical mixing body further comprises an open center, or (iv) a combination thereof.

In any aspect or embodiment described herein, one or more of the following: (i) the fluid engagement features are smooth enough to minimize cavitation or substantially eliminate cavitation, (ii) the fluid engagement features have, or the top of the cylindrical mixing body has, a substantially sinusoidal shape (for example, sinusoidal shape), (iii) the fluid engagement features create a vortex when spinning about a vertical axis, or (iv) a combination thereof.

In any aspect or embodiment described herein, the mixing apparatus further comprises an external magnetic field (ExMF) creating device that creates an ExMF that drives the mixing apparatus.

In any aspect or embodiment described herein, one or more of the following: (a) the mixing apparatus or the cylindrical mixing body is a rotary mixer; and (b) the ExMF creating device includes (i) two or more (e.g., 2, 3, 4, 5, 6, 7, 8, 9, or 10, preferably 6) field or stator coils that are sequentially energized and that are placed around the cell processing chamber (for example, the hollow cylinder of the first syringe) and in the same field as the rotatory mixer; or (ii) two or more synchronized magnets that are rotated around the cell processing chamber (for example, the hollow cylinder of the first syringe) and in the same plane as the rotary mixer.

In any aspect or embodiment described herein, the ExMF creating device includes two arms that together surround the cylindrical mixing body (for example, a container/chamber that the cylindrical mixing body is contained therein) for creating the ExMF, each arm including one or more (e.g., 1, 2, 3, or 4, preferably 3) substantially evenly spaced or substantially symmetrically located field or stator coils.

In any aspect or embodiment described herein, the two arms are articulated (e.g., articulated laterally) between a closed position that places the two arms around the cylindrical mixing body (for example, a container/chamber that the cylindrical mixing body is contained therein) for creating the ExMF and an open position that permits the cylindrical mixing body (for example, a container/chamber that the cylindrical mixing body is contained therein) to be positioned between the two arms.

In any aspect or embodiment described herein, one of the two arms of the ExMF creating device is mounted on a first body and the second of the two arms of the ExMF creating device is mounted on a second body, wherein the first body and second body are brought together (for example, through articulation between the first body and the second body, an actuator moving the first body, an actuator moving the second body, etc., or a combination thereof) such that the two arms surround the cylindrical mixing body (for example, a container/chamber that the cylindrical mixing body is contained therein) for creating the ExMF.

In any aspect or embodiment described herein, the ExMF creating device: (a) senses the position of the one or more magnets of the cylindrical mixing body or the rotary mixer relative to the field or stator coil using the back electromagnetic field (EMF) created by the permanent magnets of the rotary mixer passing by the field or stator coils; (b) continuously examines whether the cylindrical mixing body or the rotary mixer is rotating (e.g., rotating properly or as intended); (c) provides continuous intimation of how the cylindrical mixing body or the rotary mixer is rotating; or (d) a combination thereof.

In any aspect or embodiment described herein, the mixing apparatus mixes at about 500 to about 7000 rotations per minute (RPM) (for example, about 500 to about 7000 RPM, wherein the speed is increased during the mixing process, about 500 to about 3000 RPM, about 2,000 to about 7000 RPM, or about 4000 to 7000 RPM).

In any aspect or embodiment describe herein, the diverter valve (30) comprises (a) an input that splits at a single location into at least two (e.g., 2, 3, 4, 5, 6, or more) outputs (32, 35), each output including a conduit, or a region thereof, that can be reversibly pinched, compressed, or crimped, thereby stopping the flow of fluid through the output when pinched, compressed, or crimped (for example, reversibly sealable or closable); and at least one actuator or pinching member (31) that pinches the at least two outputs.

In any aspect or embodiment described herein, the at least two outputs is a first output (32) and a second output (35), and the at least one actuator or pinching member (31) is a single actuator or pinching member (31) that has (i) a first position that pinches the first output (32a) and does not pinch the second output (35), (ii) a second position that does not pinch the first output (32) or the second output (35), and, and (iii) a third position that pinches the second output (35a) and does not pinch the first output (32).

In any aspect or embodiment described herein, the single actuator or pinching member (31) is an elongated actuator or pinching member whose center point of its length is substantially located and pressed against the split without disrupting the flow when in the second position, and the elongated actuator or pinching member rotates between (i) the first position to direct the flow to the second output (35) and (ii) the third position to direct the flow to the first output (32), wherein the second position is between the first position and the second position (for example, in the unrotated state).

In any aspect or embodiment described herein, there is an actuator or pinching member (31, 245a, 245b) for each output (32, 35).

In any aspect or embodiment described herein, the at least one actuator or pinching member (31) is located just after the split resulting in zero dead volume (for example, when in a pinched, compressed, or crimped state substantially no fluid (for example, no fluid) flows into the conduit of the sealed (or closed) output).

In any aspect or embodiment described herein, the conduit or region thereof is comprised of an elastomeric material that can be reversibly pinched, compressed, or crimped, thereby stopping the flow of fluid through the output when pinched, compressed, or crimped.

An additional aspect of the present disclosure relates to a diverter valve as described herein.

A further aspect of the present disclosure relates to a method of performing nucleic acid extraction, the method comprising: (a) providing the automated nucleic extraction cartridge of the present disclosure or the automated nucleic acid extraction system of the present disclosure; (b) transferring a liquid sample from the liquid sample container into the cell processing chamber (for example, by applying a negative pressure to the cell processing chamber with the first pressure exerting device); (c) mixing the liquid sample with a processing solution in the cell processing chamber (for example, mixing with the mixing apparatus); (d) transferring the processed liquid sample from the cell processing chamber to the filter assembly (for example, by applying a positive pressure to the cell processing chamber with the first pressure exerting device); (e) washing the processed liquid sample through the filter member with a wash solution (for example, by applying a positive pressure to the wash fluid chamber comprising the wash solution), wherein the one or more pathogens are retained on the filter member and the filtrate is directed to the waste conduit by way of the diverter valve; (f) lysing the one or more pathogens through non-chemical means (for example, heating and/or sonication); and (g) isolating extracted nucleic acids from the one or more pathogens with wash solution (for example, by applying a positive pressure to the wash fluid chamber comprising the wash solution), wherein the diverter valve directs the filtrate to the pathogen nucleic acid conduit.

In any aspect or embodiment described herein, the method further comprises inserting the sampling device into the liquid sample container comprising a liquid sample (for example, placing the liquid sample container into the holder such that the sampling device is inserted into the liquid sample in the liquid sample container).

In any aspect or embodiment described herein, the method is automated (for example, after the liquid sampling container has been inserted into the holder, it is automated).

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure. Further objects, features and advantages of the disclosure will become apparent from the following detailed description taken in conjunction with the accompanying figures showing illustrative embodiments of the disclosure.

FIG. 1A. Shows an exemplary automatic nucleic acid extraction cartridge (100) of the present disclosure. The cartridge or unit (100) comprises a housing including a sample port or sampling device (2) that receives a liquid biological sample, a first syringe holder (14a), a cell processing chamber or hollow chamber of a first syringe (15), a processing port (16a) or a first pressure exerting device or a plunger of a first syringe (16), a second syringe holder (14b), a wash fluid chamber or hollow chamber of a second syringe (22), a washing fluid chamber port (23a) or a second pressure exerting device or plunger of a second syringe (23), a magnetic field window or access point (40a) for a mixing apparatus (13) located within the cell processing chamber (15), and a pathogen lysing device window or access point (40b) that facilitates its interaction with the filter assembly 21 and filter member (26). Extracted pathogen nucleic acids are deposited into the extracted nucleic acid receptacle.

FIG. 1B. Shows a top view of the exemplary automatic nucleic acid extraction cartridge (100) of FIG. 1A.

FIG. 2A. Shows a cross-sectional view of the exemplary automatic nucleic acid extraction cartridge (100) of FIG. 1B along line A-A. Shows a top view of the exemplary automatic nucleic acid extraction cartridge (100) of FIG. 1A. The cartridge or unit (100) comprises a sample port or sampling device (2) that receives a liquid biological sample in a liquid sample container (2c) and includes a sampling needle (2a), a venting needle (2b); a cell processing chamber or a cell lysis chamber (15) and first pressure exerting device (16); a wash fluid chamber (22) and a second pressure exerting device (23); and a filter assembly (21). The sample port (2) is in one-way fluid communication with the cell processing chamber (15), the cell processing chamber (15) is in one-way fluid communication with the filter assembly (21), the wash fluid chamber (22) is in fluid communication with the filter assembly (21), and the filter assembly (21) is in fluid communication with a diverter valve (30).

FIG. 2B. Shows the lower right portion of the exemplary automatic nucleic acid, as shown in the circle in FIG. 2A, extraction cartridge (100) of FIG. 2A. The cartridge or unit (100) comprises a sampling needle (2a); a cell processing chamber or a cell lysis chamber (15); a wash fluid chamber (22); and a filter assembly (21) with a filter seal (27) for a filter member (26). The sampling needle (2a) is in one-way fluid communication with the cell processing chamber (15) via a first one-way valve (3) that includes a conduit (3a) that receives a partially conical or frustoconical elastomer valve pin (3b). The cell processing chamber (15) is in one-way fluid communication with the filter assembly (21) via a first one-way valve (20) that includes a conduit (20a) that receives a partially conical or frustoconical elastomer valve pin (20b). The wash fluid chamber (22) is in fluid communication with the filter assembly (21) via a third one-way valve (30) located downstream of a plug valve/plug (24) to assure materials from the filter assembly (21) does not enter the wash fluid chamber (22). The filter assembly (21) is in fluid communication with a diverter valve (30) with a pinching member or actuator (31) and (i) a waste conduit (33) when the diverter valve (30) is biased to the first reversibly sealable (or closable) output (32) and (ii) a pathogen nucleic acid conduit (34) when the diverter valve (30) is biased to the second reversibly sealable (or closable) output (35).

FIG. 3A. Shows an exemplary external magnetic field creating device (210) having two arms (210a, 210b) that are surrounding an exemplary cell processing chamber or cell lysing chamber (15) of the automatic nucleic acid extraction cartridge (100) of FIG. 1A.

FIG. 3B. Shows a cross-sectional view of the exemplary external magnetic field creating device (210) surrounding the exemplary cell processing chamber (15) of FIG. 3A along line C-C, wherein the cell processing chamber (15) includes a mixing apparatus (13) having magnets (13b) and an open center (13d). The exemplary external magnetic field creating device (210) includes six field or stator coils (211), each wrapped around a magnetic core (212).

FIG. 4A. Shows an exemplary mixing apparatus (13) of the present disclosure with an open center (3d).

FIG. 4B. Shows a cross-sectional view of the exemplary mixing apparatus (13) of FIG. 4A along line D-D. The exemplary mixing apparatus includes a mixing body with (a) an open center, (b) magnets (13b) inserted in the mixing body, and (c) fluid engagement features that extend upward from the top of the mixing body (13a).

FIG. 4C. Shows an exemplary mixing apparatus (13) of the present disclosure that includes a mixing body with (a) an open center, (b) magnets (13b) inserted in the mixing body, and (c) fluid engagement features that extend upward from the top of the mixing body (13a).

FIG. 5A. Shows a side view of an exemplary heater (220) connected to a heater mount (221) and an exemplary sonic or ultrasonic wave transmitter (230) connected to an sonic or ultrasonic wave transmitter mount (231), the heater (220) and the wave transmitter (230) both engaged with an exemplary automatic nucleic acid extraction cartridge (100) of the present disclosure.

FIG. 5B. Shows top view of FIG. 5A.

FIG. 5C. Shows a cross-sectional view of FIG. 5B along lines F-F.

FIG. 5D. Shows a cross-sectional view of FIG. 5C along lines G-G.

FIG. 6A. Shows an exemplary diverter valve (30) having two outputs (32, 35) and a single actuator or pinching member (31) with a pivot point or point of rotation (31a) to facilitate the reversible pinching, compressing, or crimping of the outputs (32, 35). In this exemplary embodiment, the diverter valve is connected below the filter assembly (21).

FIG. 6B. Shows the exemplary diverter valve (30) of FIG. 6A in which the actuator or pinching member (31) is rotated to reversibly sealed or close output 35 (35a).

FIG. 6C. Shows the exemplary diverter valve (30) of FIG. 6A in which the actuator or pinching member (31) is rotated to reversibly sealed or close output 32 (35a).

FIG. 7A. Shows an exemplary automated nucleic acid extraction system (200) of the present disclosure comprising a system body (201) and a system door (202) that is attached to and articulates with respect to the system body (201). In this view, the system door (202) is in a closed position that creates an internal chamber that the cartridge (100) of the present disclosure is located. The exemplary door comprises a latch (203) (for example, a magnetic release) and a display (204), which may include a touchscreen input.

FIG. 7B. Shows an exemplary automated nucleic acid extraction system (200) of the present disclosure in which the system door (202) that is attached to and articulates with respect to the system body (201) is open.

FIGS. 8A, 8B, 8C, and 8D. Show an exemplary diverter valve (30) having two actuators or pinching members (245a, 245b) that independently pinches, compresses, or crimps the reversibly sealable (or closable) output (32, 35), thereby resulting in a sealed (or closed) output (32a, 35a).

FIG. 9. Shows an exemplary diverter control unit (240) to articulate an actuator or pinching member (31, 245a, 245b).

### DETAILED DESCRIPTION OF THE INVENTION

Presently described is an automated nucleic acid extraction system that permits a quick, automated method of extracting nucleic acids of one or more pathogens from a liquid sample, such as a liquid sample obtained from a subject, and associated methods. The present disclosure further provides the aspects of an automated nucleic acid extraction cartridge and an automated nucleic acid extraction instrument, each of which can be parts of the system, and facilitate the quick, automated method of extracting nucleic acids from one or more pathogens from a liquid sample, such as a liquid sample obtained from a subject.

The contents of all references, patents, pending patent applications and published patents, cited throughout this application are hereby expressly incorporated by reference.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure. In instances where a term is not specifically defined herein, that term is given an art-recognized meaning by those of ordinary skill applying that term in context to its use in describing the present disclosure.

Where a range of values is provided, it is understood that each intervening value in the range, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either/or both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element, unless otherwise indicated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, in certain methods or processes described herein that include more than one step or act, the order of the steps or acts of the method is not necessarily limited to the order in which the steps or acts of the method are recited unless the context indicates otherwise.

The term “patient” or “subject” is used throughout the specification to describe an animal, such as a mammal and preferably a human or a domesticated animal, to whom treatment, including prophylactic treatment, with the compositions according to the present disclosure is provided. In any aspect or embodiment described herein, the subject or patient is a primate. In any aspect or embodiment described herein, the patient or subject is a human. In any aspect or embodiment described herein, the patients or subjects are livestock, such as cattle, sheep, goats, cows, swine, and the like; or domesticated animals such as dogs, fish, guinea pigs, and cats. In any aspect or embodiments described herein (e.g., particularly in research contexts), subjects are rodents (e.g., mice, rats, hamsters), rabbits, primates, non-human primate, or swine, such as inbred pigs and the like. For treatment of those diseases, conditions or symptoms that are specific for a specific animal, such as a human patient, the term “patient” refers to that specific animal, including a domesticated animal such as a dog or cat, or a farm animal such as a horse, cow, sheep, etc.

General methods in molecular and cellular biochemistry can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

The present disclosure describes a disposable, single-use cartridge for extracting nucleic acids (for example, deoxyribonucleic acid (DNA) and/or ribonucleic acid (RNA)), such as nucleic acids from one or more pathogens, and an instrument configured for receiving and performing a process of extracting nucleic acids with the disposable, single-use cartridge. The automatic nucleic acid extraction cartridge of the present disclosure has a novel architecture, and the system uses novel chemical and mechanical processing techniques to extract pathogens from whole blood samples, and then extracts the DNA from those pathogens, for use in a downstream processing or assay(s) (such as, reverse transcription, polymerase chain reaction, isothermal DNA/RNA amplification, DNA/RNA sequencing, etc., for identifying one or more pathogens). The input to the system is a container (for example, a tube of venous-drawn blood, such as a 4-7 mL K2 EDTA vacuum blood collection tube, such as a Vacutainer tube) with a liquid sample (for example, whole blood) contained therein, and the output is water or buffer (for example, about 25 μL to about 200 μL or about 50 μL to 100 μL of a washing solution, such as water or a buffer) containing pathogen nucleic acids (for example, DNA), if any, in the liquid sample. In an aspect or embodiment described herein, the water or buffer containing pathogen nucleic acids is placed in a container, such as a microcentrifuge tube (for example, a 0.5-2.0 mL microcentrifuge tube, such as a 0.5 mL, 0.6 mL, 1.0 mL, 1.5 mL, 1.7 mL, or 2.0 mL microcentrifuge tube). In any aspect or embodiment described herein, the water or buffer containing pathogen nucleic acids is directly and automatically transferred to the analysis, identification, or sequencing system, without the need for a container.

Automatic Nucleic Acid Extraction Cartridge

Thus, an aspect of the present disclosure relates to an automatic nucleic acid extraction cartridge or unit The cartridge or unit (100) comprises a housing including (a) a sample port (2) that receives a liquid biological sample; (b) a cell processing chamber or a cell lysis chamber (15); (c) a wash fluid chamber (22); (d) a filter assembly (21) comprising a filter member (26); and (e) a diverter valve (30) (for example, a zero dead volume diverter valve) having a first reversibly sealable (or closable) output (32) and a second reversibly sealable (or closable) output (35), wherein (1) the sample port (2) is in one-way fluid communication with the cell processing chamber (15), (2) the cell processing chamber (15) is in one-way fluid communication with the filter assembly (21), (3) the wash fluid chamber (22) is in fluid communication (e.g., one-way fluid communication) with the filter assembly (21), and (4) the filter assembly (21) is in fluid communication (e.g., one-way fluid communication) with the diverter valve (30) and (i) a waste conduit (33) when the diverter valve (30) is biased to the first reversibly sealable (or closable) output (32) and (ii) a pathogen nucleic acid conduit (34) when the diverter valve (30) is biased to the second reversibly sealable (or closable) output (35).

In any aspect or embodiment described herein, the one or more pathogens comprises one or more bacteria, one or more virus, one or more eukaryotic pathogen, or a combination thereof.

In any aspect or embodiment described herein, the sample port (2) and the cell processing chamber (15), the cell processing chamber (15) and the filter assembly (21), the wash fluid chamber (22) and the filter assembly (21), the filter member (26) or filter assembly (21) and the diverter valve (30) are each connected via a conduit.

In any aspect or embodiment described herein, the cartridge further comprises an extracted nucleic acid receptacle (34a) that is in fluid communication with the pathogen nucleic acid conduit (34). In any aspect or embodiment described herein, the extracted nucleic acid receptacle (34a) (e.g., a microfuge tube) receives the extracted nucleic acids.

In any aspect or embodiment described herein, the cartridge further comprises a waste reservoir or tank (33a) that is in fluid communication with the waste conduit (33) or the first reversibly sealable (or closable) output (32).

In any aspect or embodiment described herein, the cartridge is provided as a kit. For example, in any aspect or embodiment described herein, the cartridge is provided as a kit and the cartridge includes a first syringe holder (14a) and a second syringe holder (14b), a first syringe (15, 16) (for example, a first syringe prefilled with processing solution), and a second syringe (22, 23) (for example, a second syringe pre-filled with wash solution), wherein the syringes are pre-installed, or installed by a user, in the first syringe holder (14a) and the second syringe holder (14b).

Sample and Sampling

In any aspect or embodiment described herein, a liquid sample container (1a) comprises a liquid sample contained therein. For example, in any aspect or embodiment described herein, the liquid sample is a blood sample.

In any aspect or embodiment described herein, the cartridge further comprises a liquid sample container holder (2c). In any aspect or embodiment described herein, the cartridge further comprises a sampling device (2) that is inserted into a liquid sample container (1a) (for example, the sampling device (2) punctures the liquid sample container (1a), such as when the liquid sample container (1a) is being placed into a liquid sample container holder (2c)).

In any aspect or embodiment described herein, the sampling device (2) is inserted into the liquid sample in an upward direction.

In any aspect or embodiment described herein, the sampling device (2) punctures the liquid sample container (1a) (e.g., the liquid sample container (1a) includes a septum (1b) that the sampling device (2) punctures and/or the sampling device (2) punctures the septum (1b) in an upward direction).

In any aspect or embodiment described herein, the sampling device (2) includes a sampling needle (2a) and a venting needle (2b), each being inserted into the liquid sample in the liquid sample container (1a) (for example, the sampling device (2) punctures the liquid sample container (1a) when being placed into the liquid sample container holder (2c) and/or inserted into the liquid sample). In any aspect or embodiment described herein, the sampling needle (2a) has (i) a larger transfer capacity than the venting needle (2b) (for example, a larger diameter when the needle has a circular cross-section or a larger side when the needle has a square or rectangular cross-section), (ii) a shorter length than the venting needle (2b) (i.e., the end of the sampling needle (2a) is located lower in the liquid sample container (1a) than the end of the venting needle (2b), preferably the sampling needle (2a) is located close to the bottom of the liquid sample container (1a), the venting needle (1b) is located close to the top of the liquid sample container (1a), or both), or (iii) both. In any aspect or embodiment described herein, the venting needle (2b) vents to the waste reservoir or tank (33a) and/or the atmosphere.

As used herein, the term “needle” includes any needle a skilled artisan would find appropriate for the described use. For example, in any aspect or embodiment described herein, the needle includes a shaft with an interior lumen that permits the flow of fluid (for example, liquid and/or gas, such as air) therethrough. For example, in any aspect or embodiment described herein, the needle includes a beveled edge and/or sharp end at the end of the shaft that is distal from the point of connection, such as connection to the sample port. The beveled edge facilitates penetration of surfaces, such as a container (1a) (for example, a liquid sample container) or septum (1b) of a container (for example, a septum (1b) of a liquid sample container (1a)). In any aspect or embodiment described herein, the liquid sample container (1a) is a vacuum blood collection tube. By way of further example, the cross-section of the shaft and/or lumen of the needle can be any appropriate shape that effectively transfers fluid, such as circular, trigonal, square, rectangular, pentagonal, hexagonal, heptagonal, octagonal, etc.

In any aspect or embodiment described herein, the liquid sample container (1a) is inserted into a holder in a downward direction so that the sampling device (2) is inserted into the liquid sample in an upward direction. As discussed herein, in any aspect or embodiment described herein, the sampling device includes a needle that is vented to the atmosphere or waste reservoir or tank (33a) (for example, a venting needle (2b)), thus allowing gas (for example, air) to flow into the liquid sample containing (for example, blood sample tube) as the liquid sample is extracted through a second needle (for example, a sampling needle (2a)). Furthermore, in any aspect or embodiment described herein, when the sampling device (2) includes two needles, the two needles may puncture the septum (1b) simultaneously, or alternatively to minimize the insertion force, it may be designed to have one needle (for example, the venting needle (2b)) penetrate the liquid sample container (1a) (for example, a septum (1b) of the container) before the second needle (for example, the sampling needle (2a)). For example, in any aspect or embodiment described herein, the venting needle (2b) penetrates the liquid sample container (1a) (for example, a septum (1b) of the container) prior to the sampling needle (2a), such that the sampling needle (2a) is located lower in the liquid sample container (1a) to facilitate the drawing of the liquid sample from the container. In any aspect or embodiment described herein, the sampling needle (2a) is located just above the internal surface of the liquid sample container (1a) or septum (1b) after the sampling device (2) is inserted into the liquid sample/liquid sampling container (1a). This helps to maximize the amount of liquid sample that can be extracted from the liquid sample container (1a) and thus processed by the cartridge/system. In any aspect or embodiment described herein, the venting needle (2b) is located just below the top of the internal surface of the liquid sample container (1a) after the sampling device (2) is inserted into the liquid sample/liquid sampling container (1a). This also helps to maximize the amount of liquid sample that can be extracted from the liquid sample container (1a).

One-Way Valves

In any aspect or embodiment described herein, the conduit connecting the sample port (2) and the cell processing chamber comprises a first one-way valve (3) (for example, a pin valve). In any aspect or embodiment described herein, the conduit of the cell processing chamber and the filter assembly (21) comprises a second one-way valve (20) (for example, a pin valve or diaphragm valve). In any aspect or embodiment described herein, the conduit of the wash fluid chamber (22) and the filter assembly (21) comprises a third one-way valve (25) (for example, a pin valve or a plug valve).

In any aspect or embodiment described herein, a one-way valve (for example, the first one-way valve (3), the second one-way valve (20), the third one-way valve (25), or a combination thereof), comprise (i) a conduit (3a, 20a) having a hollow interior that includes a straight interior region and an expanding conical interior region located at the end of the conduit that fluid exits the one-way valve (for example, the expanding conical interior region or the entire conduit can be made with an elastomeric or plastomeric material), and (ii) a partially conical elastomer valve pin or a frustoconical elastomeric valve pin (3b, 20b) (for example, a rubber pin) that mates with the expanding conical interior region of the conduit, thereby causing a substantially or completely fluid-tight (for example, liquid-tight or water-tight) seal to form when there is flow or positive pressure from a fluid contacting the larger end of the valve pin. In any aspect or embodiment described herein, the valve pin (3b, 20b) comprises an extended cylindrical area that extends from the small end of the pin and that is smaller in diameter from the straight interior region of the one-way valve. In any aspect or embodiment described herein, the taper angle of the expanding conical interior region and the valve pin (3b, 20b) is about 12 degrees to about 24 degrees. In any aspect or embodiment described herein, the valve pin (3b, 20b) has a slightly greater taper angle than the expanding conical interior region of the valve (e.g., the valve pin has a taper angle that is about 0.5 to about 1 degree greater than the expanding conical interior region of the valve).

As described herein, within the automated nucleic acid extraction cartridge, there is a passage between the sample port or the sampling device (2) and the cell processing chamber (for example, a cell lysis chamber and/or a hollow cylinder of a syringe) that includes a one-way valve (that is, the first one-way valve (3)), thus allowing the liquid sample (for example, a blood sample) to travel to the cell processing chamber, while preventing the flow of the liquid sample back into the liquid sample container (1a) (for example, a blood tube) when positive pressure is applied to the cell processing chamber (for example, a plunger for a syringe is depressed in the hollow cylinder of the syringe) at a later stage to send the processed liquid sample through the filter assembly (21). When negative pressure is applied to the cell processing chamber (for example, a plunger is withdrawn from the hollow cylinder of a syringe), such as by the instrument or system (200) described herein, the one-way valve automatically opens and the liquid sample flows from the liquid sample container (1a) into the cell processing chamber (for example, a hollow cylinder of a syringe), where the liquid sample is mixed with the processing solution (for example, a lysing solution). In any aspect or embodiment described herein, the cartridge further comprises a third one-way valve (25) (for example, a pin valve or a plug valve) connecting the wash fluid chamber (22) to the filter assembly (21). That is, the third one-way valve (25) only permits fluid movement (that is, wash solution) from the wash fluid chamber (22) to the filter assembly (21).

In any aspect or embodiment described herein, the one-way valve is a pin valve that comprises a conduit (for example, a plastic conduit) having a hollow interior that includes a straight interior region and an expanding conical interior region located at the end that fluid exits the one-way valve (for example, the expanding conical interior region or the entire conduit can be made with an elastomeric or plastomeric material), a partially conical elastomeric valve pin or a frustoconical elastomeric valve pin (for example, a rubber pin) that mates with the expanding conical interior region of the conduit, thereby causing a substantially or completely water-tight seal to form when there is flow or pressure present in the fluid coming from the large end of the valve pin (reverse flow). That is, the reverse flow causes the conical surfaces to be in contact and seal the valve, and when the flow or pressure is present at or from the small end of the pin (forward flow), the conical surface of the pin moves away from engagement with the conical surface of the conduit, thus creating a gap between the conical surfaces allowing fluid to flow in that direction. In any aspect or embodiment described herein, the valve pin further comprises an extended cylindrical area that extends from the small end of the pin and that is smaller in diameter than the straight interior region of the one-way valve. This feature aids in stabilizing the valve pin in the conduit and allows the valve pin to move more freely and remain in alignment with the conduit. The angles of the conical surfaces of the one-way pin valve are critical because if they are too small the valve pin will get jammed in the conduit of the valve and not be removable with the amount of pressure available, which is limited to below atmospheric in the case of the first one-way valve (3) described herein. If the angle of the conical portion of the pin valve is too steep the normal force between the conical sealing surfaces will be too small and it will not create a reliable seal when closed. For example, in any aspect or embodiment described herein, the taper angle of the expanding conical interior region and the valve pin is about 12 degrees to about 24 degrees. Furthermore, in any aspect or embodiment described herein, the valve pin has a slightly greater taper angle than the expanding conical interior region of the valve. This feature can further enhance the ease of which the pin valve can be opened via fluid pressure differential. That is, having a pin valve in which the valve pin has a slightly greater taper angle than the tube will allow sealing of the valve to primarily occur at the large end of the pin, thus allowing a greater release force to be created for a given pressure because the release force required is proportional to the area of the seal. In any aspect or embodiment described herein, the valve pin has a taper angle that is about 0.5 to about 1 degree greater than the expanding conical interior region of the valve.

In any aspect or embodiment described herein, the first one-way valve (3) and the cell processing chamber (for example, a hollow cylinder of a syringe) are connected via a first conduit (10) (for example, a substantially vertical tube/conduit) that is smaller than and located within a second, annular conduit (11) that connects the cell processing chamber (for example, a hollow cylinder of a syringe) and the second one-way valve (20). In any aspect or embodiment described herein, a first conduit (10) (for example, a substantially vertical conduit) connects the first one-way valve (3) and the cell processing chamber (for example, a hollow cylinder of a syringe) and a second conduit (11) connects the cell processing chamber (for example, a hollow cylinder of a syringe) and the second one-way valve (20), wherein the connections of the first conduit (10) and second conduit (11) with the cell processing chamber (for example, a hollow cylinder of a syringe) are substantially adjacent. For example, in any aspect or embodiment described herein, the first conduit (10) and the second conduit (11) share a connection (for example, a circular connection (each a semicircle of the circle), square connection (each an isosceles triangle of the square), rectangular (two congruent triangles that form a rectangle), etc.) to the cell processing chamber (for example, a hollow cylinder of a syringe). In any aspect or embodiment described herein, the first one-way valve (3) is connected at the end or bottom of the first conduit (10) (e.g., a substantially vertical conduit) and the cell processing chamber, such as a hollow cylinder of a syringe (for example, the bottom of the cell processing chamber or hollow cylinder of a syringe), is connected to the top of the first conduit (10). In any aspect or embodiment described herein, the second one-way valve (20) is connected to the end or bottom of a second conduit (11) (e.g., a substantially vertical conduit) and the cell processing chamber, such as a hollow cylinder of a syringe (for example, the bottom of the cell processing chamber, such as a hollow cylinder of a syringe), is connected to the top of the second conduit (11).

Thus, in any aspect or embodiment described herein, the configuration of the first one-way valve (3), the cell processing chamber, and the second one-way valve (20) result in the unidirectional movement of fluid from the sample port or sampling device (2) to the filter assembly (21) (for example, the filter member (26)), thereby ensuring that all fluid exiting the cell processing chamber (for example, a hollow cylinder of a syringe) is processed liquid sample and any unprocessed liquid sample has not entered the cell processing chamber, such as a hollow cylinder of a syringe (for example, in the first conduit), thereby avoiding any potential for the unprocessed liquid sample from clogging the filter member (26). Furthermore, the second one-way valve (20) prevents backward flow between the filter assembly (21) and the cell processing chamber (15), ensuring that all of fluid (such as, processed liquid sample and wash solution) passes through the filter member (26) rather than passing back into the cell processing chamber. The second one-way valve (20) also prevents remnant processed liquid sample located upstream from the second one-way valve (20) from contaminating the wash stream/solution.

Cell Processing

As used herein, the terms “cell processing chamber” and “cell processing reservoir” are used interchangeably and includes, but not limited to, the hollow cylinder of a syringe or the first syringe described herein as an exemplary cell processing chamber or reservoir and the “cell lysing chamber” as an exemplary cell processing chamber or reservoir when the processing solution is a lysis solution (for example, lyses blood cells).

In any aspect or embodiment described herein, the cell processing chamber (15) comprises a mixing apparatus (13) contained therein.

In any aspect or embodiment described herein, the cartridge further comprises a first pressure exerting device (16) in fluid communication with the cell processing chamber or lysing chamber (15). In any aspect or embodiment described herein, the first pressure exerting device (16) is capable of providing positive pressure and negative pressure to the cell processing chamber. In any aspect or embodiment described herein, the first pressure exerting device (16) moves fluid (for example, liquid sample) into the cell processing chamber when a negative pressure is applied by the first pressure exerting device (16) to the cell processing chamber. In any aspect or embodiment described herein, the first pressure exerting device (16) moves fluid (for example, processed liquid sample) into the filter assembly (21) when a positive pressure is applied by the first pressure exerting device (16) to the cell processing chamber.

As used herein, the term “first pressure exerting device” (16) includes, but is not limited to, the sliding plunger of the first syringe described herein as a specific, but not limiting, embodiment of the first pressure exerting device (16). For example, in any aspect or embodiment described herein, a non-limiting example of a first pressure exerting device (16) is a processing port that hygienically seals the cell processing chamber from the external environment and engages the first driver of the automated nucleic acid extraction system.

In any aspect or embodiment described herein, the automated nucleic acid extraction cartridge further comprises a first syringe holder (14a) that accepts a first syringe (15, 16), wherein the cell processing chamber (15) is the hollow cylinder of the first syringe and the first pressure exerting device (16) is a sliding plunger of the first syringe (for example, the first syringe holder (14a) receives the first syringe between the first one-way valve (3) and the second one-way valve (20)—upstream of the first one-way valve (3) and downstream of the second one-way valve (20)).

In any aspect or embodiment described herein, the first pressure exerting device (16) is a processing port (16a) that is capable of engaging a pressure driver from an external instrument (e.g., the first driver of the automated nucleic extraction system (200) described herein) that provides positive pressure and negative pressure to the processing port. In any aspect or embodiment described herein, the processing port (16a) seals (for example, a hermetic seal) the cell processing chamber (for example, seals the cell processing chamber from the external environment).

Processing Solution

In any aspect or embodiment described herein, the cell processing chamber (15) comprises a processing solution contained therein.

In any aspect or embodiment described herein, the processing solution is a lysis buffer that lyses non-pathogen cells (that is, the lysis buffer does not lyse the one or more pathogens). For example, in any aspect or embodiment described herein, the lyses buffer comprises a sodium hydroxide solution that is buffered to a pH of 12 using potassium chloride (KCl). In any aspect or embodiment described herein, the lysis buffer comprises 2.2% tri-sodium citrate, which helps to suppress clotting reactions. In any aspect or embodiment described herein, the liquid sample container (1a) includes K2 EDTA, which helps suppress clotting reactions. In any aspect or embodiment described herein, the lysis buffer comprises about 0.42% sodium hydroxide, about 2.7% potassium chloride, about 2.2% tri-sodium citrate, and water (for example, the remainder is waster, such as de-ionized water). In any aspect or embodiment described herein, when the liquid sample is whole blood, about 2.6 mL of processing solution or lysis buffer is used for about 4 to about 7 mL of whole blood.

In any aspect or embodiment described herein, the processing solution is a solution that does not lyse blood cells (for example, mammalian blood cells, human blood cells, etc.) and permits the processed liquid sample to pass through a sub-micron filter relatively easily, capturing the one or more intact pathogens on the filter. In any aspect or embodiment described herein, the processing solution is a solution that does not lyse red blood cells (for example, mammalian red blood cells, human red blood cells, etc.) and permits the processed liquid sample to pass through a sub-micron filter relatively easily, capturing the one or more intact pathogens on the filter. For example, in any aspect or embodiment described herein, the processing solution comprises about 0.47% sodium hydroxide dissolved in 3.3×phosphate buffered saline (PBS) buffer. In any aspect or embodiment described herein, when the liquid sample is whole blood, about 1 mL of processing solution that does not lyse blood cells (for example, red blood cells) is used for each about 1 mL of whole blood. In any aspect or embodiment described herein, when the processing solution is a solution that does not lyse blood cells (for example, red blood cells), the liquid sample-processing solution mixture is mixed for about 8 minutes to about 12 minutes (for example, about 10 minutes).

In any aspect or embodiment described herein, the processing solution makes the non-pathogenic components of the liquid sample filterable (for example, filterable without lysing eukaryotic cells, filterable by lysing non-pathogenic eukaryotic cells, etc.) without lysing one or more bacterial pathogens, one or more eukaryotic pathogens, one or more viral pathogens, or a combination thereof. In any aspect or embodiment described herein, the processing solution is a hypertonic solution relative to one or more non-pathogen eukaryotic cells of the liquid sample (for example, one or more eukaryotic cells of a subject from which the liquid sample was obtained from). In any aspect or embodiment described herein, the processing solution (i) does not lyse or break down one or more prokaryotic pathogens, (ii) does not lyse or breakdown one or more eukaryotic pathogens, (iii) does not lyse or break down one or more viral pathogens, (iv) lyses blood cells (for example, red and/or white blood cells), (v) breaks down proteins, (vi) breaks down nucleic acids, (vii) suppresses clotting, (viii) lyses non-pathogen eukaryotic cells, or (ix) a combination thereof.

Mixing Apparatus

In any aspect or embodiment described herein, the mixing apparatus (13) is a rotary mixer comprising a mixing body (13a) (for example, a plastic mixing body (13a)). In any aspect or embodiment described herein, one or more of the following: (a) the cell processing chamber includes a circular cross-section (e.g., located at the bottom of the cell processing chamber) and the rotary mixer comprises a cylindrical mixing body (13a) that rotates freely within the circular cross-section of the cell processing chamber; (b) the rotary mixer further comprises two or more (e.g., 2, 3, 4, 5, 6, 7, 8, 9, or 10, preferably 4) magnets inserted into (for example, located internally) the mixing body (13a) (for example, the rotary mixer with two or more magnets that can interact with a rotating external magnetic field, thereby causing rotation of the rotary mixer); or (c) a combination thereof.

In any aspect or embodiment described herein, the two or more magnets are substantially evenly spaced or substantially symmetrically located about the rotational axis of the rotary mixer. In any aspect or embodiment described herein, the two or more magnets have the same orientation of their polarity (i.e., the north pole of each magnet is facing outward or inward in the mixing apparatus or rotary mixer (13)). In any aspect or embodiment described herein, the mixing apparatus (13) further comprises fluid engagement features (13c) extending upward from the cylindrical mixing body (13a). In any aspect or embodiment described herein, the cylindrical mixing body (13a) further comprises an open center (13d).

In any aspect or embodiment described herein, the fluid engagement features (13c) are smooth enough to minimize cavitation or substantially eliminate cavitation. In any aspect or embodiment described herein, the fluid engagement features (13c) have, or the top of the cylindrical mixing body (13a) has, a substantially sinusoidal shape (for example, sinusoidal shape). In any aspect or embodiment described herein, the fluid engagement features (13c) create a vortex when spinning about a vertical axis.

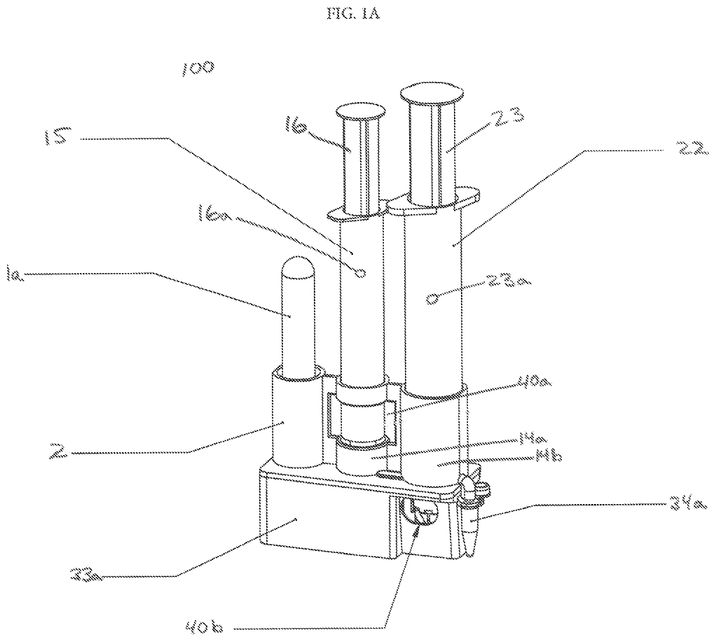

The mixing apparatus cannot practically be spun using traditional stir-plate technology, which has rotating magnets or a magnetic field directly below the mixer, in the cartridge/system of the present disclosure. For example, the connections with the cell processing chamber (for example, a hollow cylinder of a syringe) and the other features of the automated nucleic acid extraction cartridge would make it difficult to place a magnetic stir plate in a concentric axis below the mixing apparatus and close enough to the mixing apparatus to permit the mixing apparatus to be a magnetic stir-bar, especially with the embodiment of, for example, of FIG. 1. Furthermore, a standard brushless external stator (one piece) is also difficult and not workable for certain embodiments described herein, such as that depicted in FIG. 1, because it would trap the cell processing chamber (for example, a hollow cylinder of a syringe) at its center, thereby preventing or substantially complicating the separation and removal of the automated nucleic acid extraction cartridge/system of the present disclosure from the automated nucleic acid extraction system (200) disclosed herein.

Thus, in any aspect or embodiment described herein, the mixer drive system is a brushless direct current (BLDC), in-runner (field outside, magnets inside), and is current-drive or constant speed type.

In any aspect or embodiment described herein, the external electromagnetic field (ExMF) creating device (210) is capable of sensing the position of the one or more magnets of the rotary mixer relative to the field or stator coil (211) using the back electromagnetic field (EMF) created by the permanent magnets of the rotary mixer passing by the field or stator coils (211). This allows the drive circuit to precisely time the energizing of the appropriate field stator coils (211) to rotate the rotary mixer. This further permits the system to detect when the field or stator coil (211) is not correctly magnetically engaged with the magnets (13b) of the rotary mixer (for example, not correctly magnetically engaged because of an incorrectly closed door, a missing rotary mixer, a rotary mixer with missing or incorrectly installed magnets (13d), etc.). In any aspect or embodiment described herein, this feature is used to provide a continuous examination as to whether the mixing apparatus or rotary mixer (13) is rotating (e.g., rotating properly or as intended) and/or a continuous intimation of how the rotary mixer is rotating. Thus, in any aspect or embodiment described herein, the ExMF creating device (210) provides automatic and continuous confirmation of the mixing process. Standard stir-plate systems are open loop drives and thus do not provide any assurance that the mixer is actually engaged and thus mixing/spinning, and thus these existing-technology open loop systems are not suitable for automated clinical processes.

An additional aspect of the present disclosure relates to a mixing apparatus (13). For example, in any aspect or embodiment described herein, the mixing apparatus comprises a cylindrical mixing body (13a) that comprises two or more (e.g., 2, 3, 4, 5, 6, 7, 8, 9, or 10, preferably 4) magnets inserted into (for example, located internally) the cylindrical mixing body (13a).

In any aspect or embodiment described herein, one or more of the following: (i) the two or more magnets have the same orientation of their polarity (i.e., the north pole of each magnet is facing outward or inward in the mixing apparatus or rotary mixer (13)), (ii) the mixing apparatus (13) further comprises fluid engagement features (13c) extending upward from the cylindrical mixing body (13a), (iii) the cylindrical mixing body (13a) further comprises an open center (13d), or (iv) a combination thereof.

In any aspect or embodiment described herein, one or more of the following: (i) the fluid engagement features (13c) are smooth enough to minimize cavitation or substantially eliminate cavitation, (ii) the fluid engagement features (13c) have, or the top of the cylindrical mixing body (13a) has, a substantially sinusoidal shape (for example, sinusoidal shape), (iii) the fluid engagement features (13c) create a vortex when spinning about a vertical axis, or (iv) a combination thereof.

In any aspect or embodiment described herein, the mixing apparatus (13) further comprises an external magnetic field (ExMF) creating device (210) that creates an ExMF that drives the mixing apparatus (13).

In any aspect or embodiment described herein, one or more of the following: (a) the mixing apparatus (13) or the cylindrical mixing body (13a) is a rotary mixer; and (b) the ExMF creating device (210) includes (i) two or more (e.g., 2, 3, 4, 5, 6, 7, 8, 9, or 10, preferably 6) field or stator coils (211) that are sequentially energized and that are placed around the cell processing chamber (for example, the hollow cylinder of the first syringe) and in the same field as the rotatory mixer; or (ii) two or more synchronized magnets that are rotated around the cell processing chamber (for example, the hollow cylinder of the first syringe) and in the same plane as the rotary mixer.

In any aspect or embodiment described herein, the two or more field or stator coils are wound/wrapped around a magnetic core (212), such as a ferromagnetic or ferrimagnetic materials (for example, iron).

In any aspect or embodiment described herein, the ExMF creating device (210) includes two arms that together surround the cylindrical mixing body (13a) (for example, a container/chamber that the cylindrical mixing body (13a) is contained therein) for creating the ExMF, each arm including one or more (e.g., 1, 2, 3, or 4, preferably 3) substantially evenly spaced or substantially symmetrically located field or stator coils (211).

In any aspect or embodiment described herein, the two arms are articulated (e.g., articulated laterally) between a closed position that places the two arms around the cylindrical mixing body (13a) (for example, a container/chamber that the cylindrical mixing body (13a) is contained therein) for creating the ExMF and an open position that permits the cylindrical mixing body (13a) (for example, a container/chamber that the cylindrical mixing body (13a) is contained therein) to be positioned between the two arms.

In any aspect or embodiment described herein, one of the two arms of the ExMF creating device (210a or 210b) is mounted on a first body and the second of the two arms of the ExMF creating device (210a or 210b) is mounted on a second body, wherein the first body and second body are brought together (for example, through articulation between the first body and the second body, an actuator moving the first body, an actuator moving the second body, etc., or a combination thereof) such that the two arms surround the cylindrical mixing body (13a) (for example, a container/chamber that the cylindrical mixing body (13a) is contained therein) for creating the ExMF.

In any aspect or embodiment described herein, the ExMF creating device (210): (a) senses the position of the one or more magnets of the cylindrical mixing body or the rotary mixer (13a) relative to the field or stator coil (211) using the back electromagnetic field (EMF) created by the permanent magnets of the rotary mixer passing by the field or stator coils (211); (b) continuously examines whether the cylindrical mixing body or the rotary mixer (13a) is rotating (e.g., rotating properly or as intended); (c) provides continuous intimation of how the cylindrical mixing body or the rotary mixer (13a) is rotating; or (d) a combination thereof.

In any aspect or embodiment described herein, the mixing apparatus (13) mixes at about 500 to about 7000 rotations per minute (RPM) (for example, about 500 to about 7000 RPM, wherein the speed is increased during the mixing process, about 500 to about 3000 RPM, about 2,000 to about 7000 RPM, or about 4000 to 7000 RPM).

In any aspect or embodiment described herein, the mixing apparatus (13) (for example a rotary mixer) has a cylindrical mixing body (13a) with an open center (13d). For example, in any aspect or embodiment described herein, the mixing apparatus (13) has a cylindrical mixing body (13a) with an open center (13d) and a plurality (for example, 2, 3, 4, 5, 6, or more) of fluid engagement features (13c) (for example, wave-like features) extending upward from the body of the mixing apparatus (13). The fluid engagement features or wave-like features (13c) enhance the engagement of the mixer apparatus to the fluid being mixed (e.g., the processing solution with or without the liquid sample). The open center (13d) of the mixing apparatus (13) helps to minimize ventilation of the fluid being mixed. As such, the open center (13d) helps to minimize foaming of the fluid during high-speed mixing. In any aspect or embodiment described herein, the fluid engagement features (13c) are sufficiently smooth to minimize (for example, sufficiently smooth to not cause any) cavitation. Cavitation can damage the pathogens, thereby releasing their nucleic acids prematurely, allowing the nucleic acids to pass through the filter when the processed liquid sample is washed through the filter, thereby resulting in reduced, insufficient, or no pathogen nucleic acids in the nucleic acid containing wash solution to be examined, assayed, or processed. In any aspect or embodiment described herein, the mixer apparatus provides shear force sufficient to cause the eukaryotic cell (for example, blood cell) debris and human nucleic acids (such as, DNA) to be small enough to pass through the filter unimpeded with the wash solution, prior to lysis of the one or more pathogens.

Filter Assembly and Wash Fluid Chamber

As used herein, the terms “filter assembly” includes, but not limited to, the hollow cylinder of the second syringe as an exemplary filter assembly (21). In any aspect or embodiment described herein, the wash fluid chamber (22) comprises a wash solution (for example, a wash buffer, such as phosphate buffered saline (PBS) or water)) contained therein.

In any aspect or embodiment described herein, the cartridge further comprises a second pressure exerting device (23) in fluid communication with the wash fluid chamber (22). As used herein, the term “second pressure exerting device” includes, but is not limited to, the sliding plunger of a syringe as a specific, but not limiting, embodiment of the second pressure exerting device (23). In any aspect or embodiment described herein, the automated nucleic acid extraction cartridge further comprises a second syringe holder (14b) that accepts a second syringe (22, 23), wherein the wash fluid chamber (22) is a hollow cylinder of the second syringe and the second pressure exerting device (23) is a sliding plunger of the second syringe.

In any aspect or embodiment described herein, a non-limiting example of a second pressure exerting device (23) is a wash fluid chamber port (23a) that is capable of engaging a pressure driver from an external instrument (for example, the second driver (250b) of the automatic nucleic acid extraction system (200) described herein) that provides at least positive pressure to the wash fluid chamber port. For example, in any aspect or embodiment described herein, the wash fluid chamber port (23a) hygienically seals the wash fluid chamber (22) from the external environment.

In any aspect or embodiment described herein, the second pressure exerting device (23) is capable of providing at least positive pressure to the wash fluid chamber (22). In any aspect or embodiment describe herein, the extracted nucleic acid receptacle (34a) (e.g., a microfuge tube) receives the extracted nucleic acids when the second pressure exerting device (23) passes a portion of the wash solution through the filter assembly (21) after the one or more pathogens are lysed from the second reversibly sealable (or closable) output (35).

In any aspect or embodiment described herein, the filter assembly (21) further comprises a thermally conductive element (for example, a thermally conductive metal element) that transmits thermal energy to the filter member (26). In any aspect or embodiment described herein, the filter assembly (21) further comprises a metal element that transmits at least vibrational energy (for example, sound waves, sonic energy, ultrasonic energy, etc.) to the filter member (26) (for example, the metal element transmits both vibrational energy and thermal energy to the filter member (26)).

In any aspect or embodiment described herein, the filter assembly (21) further comprises a filter housing (26a) that receives the filter member (26), which optionally includes a filter seal (27) as described herein. In any aspect or embodiment described herein, the filter assembly (21) further comprises a filter seal (27) (e.g., an elastomeric seal or rubber seal) that provides a fluid tight seal between the filter assembly and the filter member (26). For example, in any aspect or embodiment described herein, the filter seal (27) is an elastomeric seal. In any aspect or embodiment described herein, the filter seal (27) is an O-ring or any other shape the filter member (26) may take (for example, square, rectangular, circular, oval, pentagonal, hexagonal, etc.).

In any aspect or embodiment described herein, the second pressure exerting device (23) is a wash fluid chamber port (23a) that is capable of engaging a pressure driver from an external instrument (e.g., the second driver (250b) of the automated nucleic extraction system (200) described herein) that provides at least positive pressure to the wash fluid chamber port (23a). In any aspect or embodiment described herein, the wash fluid chamber port (23a) seals (for example, a hermetic seal) the wash fluid chamber (22) from the external environment.

Filter Member. In any aspect or embodiment described herein, after the solution in the cell processing chamber (for example, a syringe) is sufficiently or completely mixed, the solution is then driven through a filter member (26) to capture the still-intact pathogens. In any aspect or embodiment described herein, the filter member (26) is configured to retain one or more pathogens. For example, in any aspect or embodiment described herein, the filter member (26) retains one or more pathogens when the second pressure exerting device (23) applies a positive pressure to the wash fluid chamber (22), thereby passing a portion of the wash solution through the filter assembly (21). For example, in any aspect or embodiment described herein, the filter member (26) has pores that are smaller than the smallest pathogen that needs to be captured, which is a pore size of 0.4 microns for bacterium (for example, Staphylococcus), 0.2 microns for viruses, and 3 microns for fungi.

In any aspect or embodiment described herein, the filter member (26) has a pore size that retains one or more pathogens (for example, a pore size of about 0.45 μm or less—such as, about 0.45 μm, about 0.4 μm, about 0.3 μm, about 0.22 μm, about 0.2 μm, or about 0.1 μm). In any aspect or embodiment described herein, the filter member (26) has a coating (e.g., a composition, a ligand, a peptide, an antibody, etc.) that has an affinity for or captures one or more pathogens. In any aspect or embodiment described herein, the filter member (26) is statically charged. In any aspect or embodiment described herein, the filter member (26) is made of a material that does not induce a fibrinogen-driven clotting reaction (for example, the filter member (26) is a polycarbonate filter or a polyester filter).

In any aspect or embodiment described herein, the filter member (26) is a Track-Etched filter. Track-Etched filters provide pores with simple cylindrical geometry, accurate pore sizing, and a high enough open area ratio for reasonable filter pressures of less than 300 pounds per square inch (psi) for reasonable filter times of less than one minute. In any aspect or embodiment described herein, the filter member (26) is a circular filter member—for example, a circular filter member with an active area that has a diameter of about 6 mm to about 25 mm (for example, about 6 mm to about 12 mm or about 9 mm).

In any aspect or embodiment describe herein, the diverter valve (30) comprises (a) an input that splits at a single location into at least two (e.g., 2, 3, 4, 5, 6, or more) outputs (32, 35), each output including a conduit, or a region thereof, that can be reversibly pinched, compressed, or crimped, thereby stopping the flow of fluid through the output when pinched, compressed, or crimped (for example, reversibly sealable or closable); and at least one actuator or pinching member (31) that pinches the at least two outputs.

In any aspect or embodiment described herein, the at least two outputs is a first output (32) and a second output (35), and the at least one actuator or pinching member (31) is a single actuator or pinching member (31) that has (i) a first position that pinches the first output (32a) and does not pinch the second output (35), (ii) a second position that does not pinch the first output (32) or the second output (35), and, and (iii) a third position that pinches the second output (35a) and does not pinch the first output (32).

In any aspect or embodiment described herein, the single actuator or pinching member (31) is an elongated actuator or pinching member whose center point of its length is substantially located and pressed against the split without disrupting the flow when in the second position, and the elongated actuator or pinching member rotates between (i) the first position to direct the flow to the second output (35) and (ii) the third position to direct the flow to the first output (32), wherein the second position is between the first position and the second position (for example, in the unrotated state).

In any aspect or embodiment described herein, there is an actuator or pinching member (31, 245a, 245b) for each output (32, 35).

In any aspect or embodiment described herein, the at least one actuator or pinching member (31) is located just after the split resulting in zero dead volume (for example, when in a pinched, compressed, or crimped state substantially no fluid (for example, no fluid) flows into the conduit of the sealed (or closed) output).

In any aspect or embodiment described herein, the conduit or region thereof is comprised of an elastomeric material that can be reversibly pinched, compressed, or crimped, thereby stopping the flow of fluid through the output when pinched, compressed, or crimped.

In any aspect or embodiment described herein, the diverter valve (30) (for example, a zero dead volume diverter valve) includes a first reversibly sealable (or closable) output (32) and a second reversibly sealable (or closable) output (35), wherein (i) when the diverter valve (30) is biased to the first reversibly sealable (or closable) output (32) fluid is directed to a waste conduit (33), and (ii) when the diverter valve (30) is biased to the second reversibly sealable (or closable) output (35) the fluid is directed to a pathogen nucleic acid conduit (34). In any aspect or embodiment described herein, the first reversibly sealable (or closable) output (32) includes a region that can be reversibly pinched, compressed, or crimped to be in a sealed (or closed) position that does not allow fluid to pass through. In any aspect or embodiment described herein, the second reversibly sealable (or closable) output (35) includes a region that can be reversibly pinched, compressed, or crimped to be in a sealed (or closed) position that does not allow fluid to pass through. In any aspect or embodiment described herein, the first reversibly sealable (or closable) output (32) and the second reversibly sealable (or closable) output (35) are both in a sealed (or closed) position.

In any aspect or embodiment described herein, the diverter valve (30) directs the filtrate exiting the filter member (26) to either a waste conduit/tube (33) or a pathogenic nucleic acid conduit/tube (or output conduit/tube) (34). In any aspect or embodiment described herein, the waste conduit (33) exits to an external waste container or tank (for example, part of the instrument) or an internal waste container or tank that is part of the automated nucleic acid extraction cartridge. In any aspect or embodiment described herein, the pathogen nucleic acid conduit (34) exits to an extracted nucleic acid receptacle or tube (34a) for receiving the extracted pathogen nucleic acids. In any aspect or embodiment described herein, the cartridge further comprises a conduit/passage/tube that provides fluid communication from the filter member (26) to the diverter valve (30).

In any aspect or embodiment described herein, the diverter valve (30) further comprises an actuator or pinching member (245a) for the waste conduit/tube (33) and an actuator or pinching member (245b) for the pathogenic nucleic acid conduit/tube (or output tube/conduit). The use of multiple actuators or pinching members (31, 245a, 245b) in the diverter valve (30) (for example, an actuator or pinching member for each output of the diverter valve (30)) provides the advantage of allowing each exit path (for example, two exit paths) to be closed at the same time. This would be particularly advantageous when using a sonic or ultrasonic pathogen lysing method, as described herein, since it prevents the fluid being treated in the filter assembly (21) from traveling downstream in the event that the fluid temperature becomes too high and the vapor pressure is sufficient to cause some of the fluid to pass through the filter member (26) prior to completion of lysing the one or more pathogens.

In any aspect or embodiment described herein, the cartridge comprises a one-way valve (25) (for example, a third one-way valve) between the wash fluid chamber (22) and the filter assembly (21). The third one-way valve (25) prevents any backflow into the wash fluid chamber (22) when the process liquid sample is passed through the filter member (26) by the first pressure exerting device (16) exerts a positive pressure to the filter assembly (21). The third-one-way valve also prevents flow, and thus diffusion of nucleic acids, upstream into the wash fluid chamber (22) (for example, a syringe) during and after lysing the one or more pathogens. Thus, the third one-way valve (25) may increase the concentration of nucleic acids in the wash solution that exits the cartridge (for example, exit through the pathogenic nucleic acid conduit). In any aspect or embodiment described herein, filter assemble is a hollow cylinder of a syringe, which optionally includes a one-way valve in the tip of the syringe (for example, a one-way valve on the Luer fitting of the hollow cylinder of a syringe).

Furthermore, in any aspect or embodiment described herein, the cartridge comprises a plug valve (24) immediately after the wash fluid chamber (22). In any aspect or embodiment described herein, the plug (24) is a small elastomeric fitting (for example, a silicone plug) that plugs the opening of the wash fluid chamber (22) or a conduit between the wash fluid chamber (22) and the filter assembly (21) (for example, a plug (24) that plugs a male Luer fitting on the hollow cylinder of a syringe), and that is displaced (for example, automatically forced off) when the second pressure exerting device (23) applies a positive pressure to the wash fluid chamber (22). In any aspect or embodiment described herein, the plug (24) of the plug valve has outer groove features in the external, downstream side of the plug (24) that prevents the plug (24) from plugging the downstream flow path when it is displaced or released from the opening or conduit (for example, an exit of a hollow cylinder of a syringe).

Lysing Pathogen(s) through Sonication. In any aspect or embodiment described herein, one or more pathogens located on the filter member (26) are lysed by sonication. For example, in any aspect or embodiment described herein, wash solution containing the one or more pathogens is heated to a temperature of about 65° C. to about 100° C. and sonicated. For example, in any aspect or embodiment described herein, the one or more pathogens are sonicated for about 15 seconds to about 5 minutes (for example, about 15 seconds to 4 minutes, about 15 seconds to about 3 minutes, or about 15 seconds to about 2 minutes). In any aspect or embodiment describe herein, sonication includes transmitting sounds waves of 20,000 kHz or greater. The lysing of the one or more pathogens results in the release of the pathogen(s) nucleic acids (for example, DNA and/or RNA) into the surrounding solution.

Lysing Pathogen(s) through Superheating. A further advantage of being able to close off the exit paths or outputs (for example, the waste conduit/tube (33) and/or the pathogen nucleic acid conduit/tube (34)) from the filter assembly (21)/diverter valve (30) is that it allows the pathogens to be lysed by superheating. Thus, in any aspect or embodiment described herein, the exit paths or outputs (for example, the waste conduit/tube (33) and the pathogen nucleic acid conduit/tube (34)) from the filter assembly (21)/diverter valve (30) are closed, and wash solution containing the one or more pathogens is heated a temperature that will lyse the one or more pathogens (e.g., above 100° C.) and held while at least some (for example, all of the pressure) of the elevated pressure in the filter assembly (21) caused by the heating is maintained. In any aspect or embodiment described herein, the pressure is quickly released by opening the diverter valve (30) to the waste conduit/tube (33). The quick release of the pressure at the elevated temperature results in the fluid contained in and/or around the one or more pathogens (e.g., prokaryotic or eukaryotic pathogen cell(s) or virion(s)) to boil, thereby rupturing the one or more pathogens, release their nucleic acids (for example, DNA and/or RNA) into the surrounding solution. In any aspect or embodiment described herein, superheating lysing method is combined with sonic or ultrasonic lysing to ensure that very difficult to lyse pathogens (for example, fungal cells) are fully lysed.

Automated Nucleic Acid Extraction Instrument and Methods of Using the Same

A further aspect of the present disclosure is an automated nucleic acid extraction system (200) comprising the automated nucleic acid extraction cartridge (100) of the present disclosure and a system body (201) that comprises: (a) a first driver (250a) that provides the force to the first pressure exerting device (16) to exert the negative pressure to the cell processing chamber and the positive pressure to the cell processing chamber; (b) a second driver (250b) that provides the force to the second pressure exerting device (23) to exert a positive pressure to the wash fluid chamber (22); (c) an external magnetic field (ExMF) creating device (210) that creates an ExMF that drives the mixing apparatus (13); (d) a pathogen lysing device (220, 230) that engages the filter assembly (21) (for example, the filter member (26)) and that lyses the one or more pathogens contained in the filter assembly (21) (for example, on the filter member (26)); and (e) a diverter valve control unit (240) that controls the diverter valve (30).

In any aspect or embodiment described herein, the first position of each of the actuator or pinching member (31) closes the output by pinching, compressing, or crimping a region of the output that can be reversibly pinched, compressed, or crimped.

In any aspect or embodiment described herein, the system body (201) further comprises a control unit that controls the first drive (250a), the second driver (250b), the ExMF creating device (210), the pathogen lysing device (220, 230), the diverter valve (30), or a combination thereof. In any aspect or embodiment described herein, the system (200) further comprises a system door (202) that is attached to and articulated with the system body (201) between an open position in which the cartridge is accessible and a closed position that produces an enclosed space where the cartridge is placed or located.

In any aspect or embodiment described herein, the mixing apparatus (13) is a rotary mixer; and the ExMF creating device (210) includes (i) two or more (e.g., 2, 3, 4, 5, 6, 7, 8, 9, or 10, preferably 6) field or stator coils (211) that are sequentially energized and that are placed around the cell processing chamber (for example, the hollow cylinder of the first syringe) and in the same field as the rotatory mixer; or (ii) two or more synchronized magnets that are rotated around the cell processing chamber (for example, the hollow cylinder of the first syringe) and in the same plane as the rotary mixer.

In any aspect or embodiment described herein, the ExMF creating device (210) includes two arms (210a, 210b) that together surround the cell processing chamber (15) (for example, the hollow cylinder of the first syringe) for creating the ExMF, each arm including one or more (e.g., 1, 2, 3, or 4, preferably 3) substantially evenly spaced or substantially symmetrically located field or stator coils (211).

In any aspect or embodiment described herein, the two arms are articulated (e.g., articulated laterally) between a closed position that places the two arms around the cell processing chamber (for example, the hollow cylinder of the first syringe) for creating the ExMF and an open position that permits the cell processing chamber to be positioned between the two arms.

In any aspect or embodiment described herein, one of the two arms of the ExMF creating device (210b) is mounted on the system body (201) and the second of the two arms of the ExMF creating device (210a) is mounted on the system door (202).

In any aspect or embodiment described herein, the ExMF creating device (210) senses the position of the one or more magnets of the rotary mixer relative to the field or stator coil (211) using the back electromagnetic field (EMF) created by the permanent magnets of the rotary mixer passing by the field or stator coils (211). In any aspect or embodiment described herein, the ExMF creating device (210) continuously examines whether the rotary mixer is rotating (e.g., rotating properly or as intended). In any aspect or embodiment described herein, the ExMF creating device (210) provides continuous intimation of how the rotary mixer is rotating.

In any aspect or embodiment described herein, the mixing apparatus (13) mixes at about 500 to about 7000 rotations per minute (RPM). For example, in any aspect or embodiment described herein, the mixing apparatus (13) mixes at about 500 to about 7000 RPM, wherein the speed is increased during the mixing process, about 500 to about 3000 RPM, about 2,000 to about 7000 RPM, or about 4000 to 7000 RPM. In any aspect or embodiment described herein, the mixing apparatus (13) mixes at least while the liquid sample is introduced into the cell processing chamber. In any aspect or embodiment described herein, the mixing apparatus (13) mixes the liquid sample and the processing solution for about 3 to about 10 minutes.

In any aspect or embodiment described herein, the diverter valve control unit (240) comprises an actuator or pinching member (31) that has (i) a first position that pinches, compresses, or crimps (that is, a sealed or closed position) for the first reversibly sealable (or closable) output, (ii) a second position that pinches, compresses, or crimps (that is, a sealed or closed position) for the second reversibly sealable (or closable) output (35), and (iii) a third position that does not pinch, compress, or crimp either of the first or the second reversibly sealable (or closeable) outputs to the point of stopping the flow of fluid (that is, an unsealed or opened position).

In any aspect or embodiment described herein, the diverter valve control unit (240) comprises a first actuator or pinching member (245a) for the first reversibly sealable (or closable) output (32) and a second actuator or pinching member (245b) for the second reversibly sealable (or closable) output (35), wherein each actuator (245a, 245b) has a first position that seals (or closes) the output (32b, 35b) and a second position that opens the output (32a, 35b).

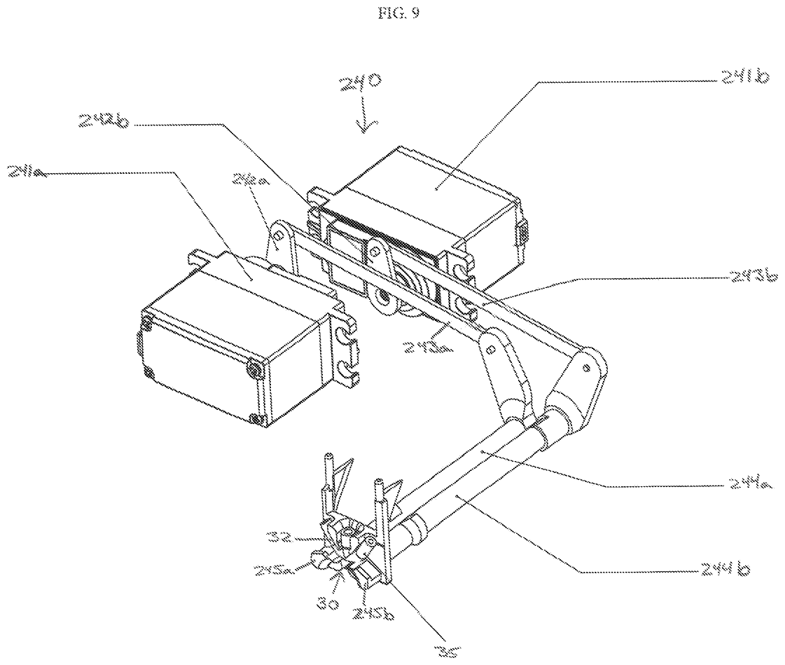

FIG. 9 illustrates an exemplary diverter control unit (240) to articulate an actuator or pinching member (31, 245a, 245b). For example, in any aspect or embodiment described therein, the first actuator or pinching member (245a) is articulated via first servo motor (241a) via or with a first servo arm (242a). Similarly, in any aspect or embodiment described herein, the second actuator or pinching member (245b) is articulated via a second servo motor (241b) via or with a second servo arm (242b). For example, in any aspect or embodiment described herein, the first servo arm (242a) may be connected or linked to a first actuator or pinching member (245a, 245b) via a first rotating actuator shaft (244a). Similarly, in any aspect or embodiment described herein, the second servo arm (242b) may be connected or linked to a second actuator or pinching member (245b) via a second rotating actuator shaft (244b).

Furthermore, in any aspect or embodiment described herein, the first rotating actuator shaft (244a) is connector or linked to the servo motor arm (242a) via a first servo shaft (243a). Similarly, in any aspect ore embodiment described herein, the second rotating actuator shaft (244a) is connector or linked to the second servo motor arm (242b) via a first servo shaft (243a).

In any aspect or embodiment described herein, the pathogen lysing device (220, 230) comprises (a) a heater (220) that heats contents of the filter assembly (21) to a temperature sufficient to lyse the one or more pathogens (for example, at least 100° C.); (b) a sonic or ultrasonic wave transmitter (230) (for example, sound waves of 20,000 kHz or greater) that transmits sound waves (for example, sonic or ultrasonic energy) to contents of the filter assembly (21) (for example, for about 15 seconds to about 5 minutes) that are sufficient to lyse the one or more pathogens; or (c) a combination thereof.

In any aspect or embodiment described herein, one or more of the following: (a) the first driver (250a) further comprises at least one force sensor that detects how much force is being exerted on the cell processing chamber, (b) the second driver (250b) further comprises at least one force sensor that detects how much force is being exerted on the wash fluid chamber (22), or (c) a combination thereof.

In any aspect or embodiment described herein, the system body (201) further comprises a waste reservoir or tank (33a) that is in fluid communication with (that is, receives fluid from) the end of the waste conduit (33) not connected to the diverter valve (30) (for example, the automated nucleic acid extraction system (200) does not contain a waste reservoir or tank (33a)).

In any aspect or embodiment described herein, the first driver (250a), the second driver (250b), or both, includes at least one force sensor (for example, a compressive force sensor) that detects how much force is being exerted on the cell processing chamber (15) and/or a wash fluid chamber (22) by way of the first pressure exerting device (16) and/or the second pressure exerting device (23). In any aspect or embodiment described herein, the force (for example, compressive force) detected is transmitted to the control unit. Thus, in any aspect or embodiment described herein, the instrument or control unit described herein can determine the amount of hydrostatic pressure in the cartridge, such as in each portion of the cartridge, described herein as any time. This information allows the instrument, control unit, or methods described herein to be operated/performed at the maximum safe speed for the system, thus completing runs in the shorted amount of time. Also, the safety of the system is increased because the force signals from the at least one force sensor can be used to prevent the cartridge or system from being over-pressurized. In any aspect or embodiment described herein, this information can also be used to sense when the cartridge or system is under-pressure, thereby detecting a problem/fault with the cartridge or system, such as, a faulty filter assembly, a faulty filter member, or a leak in the cartridge or system.

In any aspect or embodiment described herein, the instrument comprises an instrument door that articulates between an open position in which the cartridge is accessible and a closed position that produces an enclosed space where the cartridge is located or placed.

In any aspect or embodiment described herein, all or substantially all of the processed liquid sample is expelled from the filter assembly (21)/filter member (26). For example, the first pressure exerting device (16) (for example, a sliding plunger of a syringe) and/or the first driver (250a) expels all or substantially all of the processed liquid sample from the cell processing chamber after mixing.

In any aspect or embodiment described herein, after the processed liquid sample enters the filter assembly (21) via the second one-way valve (20), a portion of the wash solution is passed through the filter member (26) (that is, by injection into the filter assembly (21)) and the solution directed to the waste conduit/tube (33) by way of the diverter valve (30). For example, the second pressure exerting device (23) (for example, a sliding plunger of a syringe) and/or the second driver (250b) expels a portion of the wash solution through the filter member (26) (for example, by injection into the filter assembly (21)) and the solution is directed to the waste conduit/tube (33) by way of the diverter valve (30). This results in the second one-way valve (20) to close and all of the wash solution to flow through to the waste conduit/tube (33). At this stage, a portion of the wash solution is still present in the wash fluid chamber (22) and in the filter assembly (21) (for example, on the filter member (26)).

In any aspect or embodiment described herein, after the washing of the processed liquid sample, the diverter valve (30) directs the filtrate to the pathogenic nucleic acid conduit/tube (or closes all outputs of the diverter valve (30)) and the one or more pathogens are lysed (for example, lysed as described herein). After pathogen lysing, the nucleic acids of the pathogen(s) are in in the wash solution present.

In any aspect or embodiment described herein, after the one or more pathogens are lysed, a portion of the wash solution is passed through the filter (i.e., by injection into the filter assembly (21)) so that about 25 μL to about 200 μL (for example, about 50 μL to about 200 μL or about 50 μL to 100 μL) of wash solution comprising nucleic acids from the one or more pathogens is directed through and out of the pathogen nucleic acid conduit/tube (34) by way of the diverter valve (30). For example, the second pressure exerting device (23) (for example, a sliding plunger of a syringe) and/or the second driver (250b) expels a portion of the wash solution through the filter member (26) (for example, by injection into the filter assembly (21)) so that about 50 μL to about 200 μL of wash solution comprising nucleic acids from the one or more pathogens is directed through and out of the pathogen nucleic acid conduit/tube (34) by way of the diverter valve (30). In any aspect or embodiment described herein, the wash solution comprising nucleic acids from the one or more pathogens is placed in an extracted nucleic acid receptacle or tube (34a), such as a microfuge tube. The passing of the wash solution comprising nucleic acids from the one or more pathogens through the pathogen nucleic acid conduit/tube (34) completes the extraction process, and if not already done, the extracted nucleic acid receptacle (34a) (for example, a microfuge tube) is removed from the cartridge or instrument. The extracted nucleic acids can be frozen or refrigerated for future analysis or immediately analyzed/examined or further processed via any appropriate assay or technique for nucleic acids (for example, reverse transcription, polymerase chain reaction, isothermal DNA/RNA amplification, DNA/RNA sequencing polymerase chain reaction, etc.).

For sufficient lysing of the one or more pathogens, the volume of fluid or wash solution surrounding the filter member (26) needs to be in intimate physical contact with a source of accurately controlled heat and sonic or ultrasonic energy, as described herein. In any aspect or embodiment described herein, the force needed to maintain intimate contact between these surfaces is provided when a door to the instrument is closed around the cartridge. In any aspect or embodiment described herein, after the cartridge is inserted into the instrument, (i) the external magnetic field (ExMF) creating device (210) is in position to rotate the rotary mixer, and (ii) the pathogen lysing device (220, 230) is in place to lyse the one or more pathogens located on the filter member (26) and/or in the wash solution. This can be accomplished by any appropriate means. For example, in any aspect or embodiment described herein, the insertion of the cartridge results in the appropriate placement of the external magnetic field (ExMF) creating device (210) and/or in the appropriate placement of the pathogen lysing device (220, 230) to lyse the one or more pathogens. By way of further example, after the cartridge is inserted into the instrument, the motion of closing a door of the instrument results in the appropriate placement of the external magnetic field (ExMF) creating device (210) and/or in the appropriate placement of the pathogen lysing device (220, 230) to lyse the one or more pathogens. For example, this can be accomplished through an automated system (for example, one or more actuators) that is triggered by the closing of the door or can be accomplished through a mechanical system that is driven by the closing of the door.

In any aspect or embodiment described herein, the heater of the pathogen lysing device (220, 230) is mounted on a door of the instrument so that when the door is closed it is sufficiently located to heat the one or more pathogens and wash solution on the filter member (26). In any aspect or embodiment described herein, the sonic or ultrasonic wave transmitter (230) of the pathogen lysing device (220, 230) is mounted on a door of the instrument so that when the door is closed it is sufficiently located to transmit sound waves to the one or more pathogens and wash solution on the filter member (26).

In any aspect or embodiment described herein, the heater (220) of the pathogen lysing device (220, 230) is mounted on the instrument so that it is sufficiently located to heat the one or more pathogens and wash solution on the filter member (26) when the cartridge is inserted into the instrument. In any aspect or embodiment described herein, the sonic or ultrasonic wave transmitter (230) of the pathogen lysing device (220, 230) is mounted on the instrument such that it is sufficiently located to transmit sound waves to the one or more pathogens and wash solution on the filter member (26) when the cartridge is inserted into the instrument.

In any aspect or embodiment described herein, the external magnetic field (ExMF) creating device (210) includes two arms (210a, 210b) that together surround the cell processing chamber or cylinder of the first syringe for creating the ExMF, each arm including one or more (e.g., 1, 2, 3, or 4, preferably 3) substantially evenly spaced or substantially symmetrically located field or stator coils (211). For example, in any aspect or embodiment described herein, the two arms are articulated (e.g., articulated laterally) between a closed position that places the two arms around the cell processing chamber or cylinder of the first syringe for creating the ExMF and an open position that permits the cell processing chamber or cylinder of the first syringe to be positioned between the two arms. By way of further example, in any aspect or embodiment described herein, the instrument further comprises an instrument door that articulates between an open position in which the cartridge is accessible and a closed position that produces an enclosed space where the cartridge is placed or located, wherein one of the two arms of the ExMF creating device (210b) is mounted on the body (201) of the instrument (200) and the second of the two arms of the ExMF creating device (210a) is mounted on the instrument door (202)).

In any aspect or embodiment described herein, the instrument further comprises one or more optical sensors. For example, the optical sensors can discriminate between the liquid sample (for example, blood) or processed liquid sample (for example, lysed blood), from the wash solution or no solution in any portion of the cartridge and/or instrument (for example, the cell processing chamber, the filter assembly (21), the waste container or tank, a conduit, a tube, a passage, the nucleic acid collection tube, etc.). The use of sensors, while not required, would allow the instrument to confirm the transfer of the liquid sample in and out of the cell processing chamber, thus providing assurance that the output of the cartridge will contain any pathogen nucleic acid present in the liquid sample, and thus prevent false negative results. In any aspect or embodiment described herein, the instrument includes (i) a pressure exerting device, such as a sliding plunger of a syringe, sensor that detects/senses when no pressure has been exerted, and/or (ii) a pressure exerting device, such as a sliding plunger of a syringe, completion sensor that detects/senses when it has completed exerting pressure. Having one or more of the pressure exerting device sensors provides further confirmation that the pressure exerting device is functioning properly, which also allows homing the cartridge/system prior to each sample run. In any aspect or embodiment described herein, the instrument comprises a door sensor and/or interlock, in order to detect or prevent the door from being open when processing a sample. Opening the door while processing a sample would physically and magnetically disengage the external magnetic field (ExMF) creating device (210) and disengage the pathogen lysing device (220, 230) from the filter assembly (21)/filter member (26), possibly affecting the result of the process, depending on the step being performed when the door is opened.

In any aspect or embodiment described herein, a positive control pathogen is used (for example, inserted into the liquid sample container (1a) prior to processing, is present in the sampling unit in a tube/conduit connecting the sampling device (2) and the first one-way valve (3), etc.).

In any aspect or embodiment described herein, the wash solution removes substantially all contaminants that would inhibit analysis or further processing of the extracted nucleic acids (for example, reserves transcription, polymerase chain reaction, etc.) when the processed liquid sample is passed through the filter member (26).

In any aspect or embodiment described herein, the wash solution removes substantially all subject nucleic acids present in the sample when the processed liquid biological sample is passed through the filter member (26). For example, in any aspect or embodiment described herein, the wash solution removes at least 90% (for example, at least 92%, at least 93%, at least 94%, at least 95%, at least 96%, at least 97%, at least 98%, at least 99%, at least 99.5%, or at least 99.9%) of the subject's nucleic acids (for example, DNA and/or RNA) that is present in sample when the processed liquid biological sample is passed through the filter member (26).

In any aspect or embodiment described herein, mixing starts at a lower speed and increases as the mixture becomes less viscous. For example, in any aspect or embodiment described herein, mixing the liquid biological sample-processing solution mixture starts at about 500 to about 3000 RPM and increased to about 4000 to about 7000 RPM.

Method of Extracting Nucleic Acids

A further aspect of the present disclosure relates to a method of performing nucleic acid extraction, the method comprising: (a) providing the automated nucleic extraction cartridge (100) of the present disclosure or the automated nucleic acid extraction system (200) of the present disclosure; (b) transferring a liquid sample from the liquid sample container (1a) into the cell processing chamber (for example, by applying a negative pressure to the cell processing chamber with the first pressure exerting device (16)); (c) mixing the liquid sample with a processing solution in the cell processing chamber (for example, mixing with the mixing apparatus (13)); (d) transferring the processed liquid sample from the cell processing chamber to the filter assembly (21) (for example, by applying a positive pressure to the cell processing chamber with the first pressure exerting device (16)); (e) washing the processed liquid sample through the filter member (26) with a wash solution (for example, by applying a positive pressure to the wash fluid chamber (22) comprising the wash solution), wherein the one or more pathogens are retained on the filter member (26) and the filtrate is directed to the waste conduit (33) by way of the diverter valve (30); (f) lysing the one or more pathogens through non-chemical means (for example, heating and/or sonication); and (g) isolating extracted nucleic acids from the one or more pathogens with wash solution (for example, by applying a positive pressure to the wash fluid chamber comprising the wash solution), wherein the diverter valve (30) directs the filtrate to the pathogen nucleic acid conduit (34).

In any aspect or embodiment described herein, the method further comprises inserting the sampling device (2) into the liquid sample container (1a) comprising a liquid sample (for example, placing the liquid sample container (1a) into the holder such that the sampling device (2) is inserted into the liquid sample in the liquid sample container (1a)).

In any aspect or embodiment described herein, the method is automated (for example, after the liquid sampling container has been inserted into the holder, it is automated).

### EXEMPLARY EMBODIMENTS OF THE PRESENT DISCLOSURE

FIG. 1A shows an exemplary automatic nucleic acid extraction cartridge (100) of the present disclosure. The cartridge or unit (100) comprises a housing including a sample port or sampling device (2) that receives a liquid biological sample, a first syringe holder (14a), a cell processing chamber or hollow chamber of a first syringe (15), a processing port (16a) or a first pressure exerting device or a plunger of a first syringe (16), a second syringe holder (14b), a wash fluid chamber or hollow chamber of a second syringe (22), a washing fluid chamber port (23a) or a second pressure exerting device or plunger of a second syringe (23), a magnetic field window or access point (40a) for a mixing apparatus (13) located within the cell processing chamber (15), and a pathogen lysing device window or access point (40b) that facilitates its interaction with the filter assembly 21 and filter member (26). Extracted pathogen nucleic acids are deposited into the extracted nucleic acid receptacle. FIG. 1B shows a top view of the exemplary automatic nucleic acid extraction cartridge (100) of FIG. 1A.

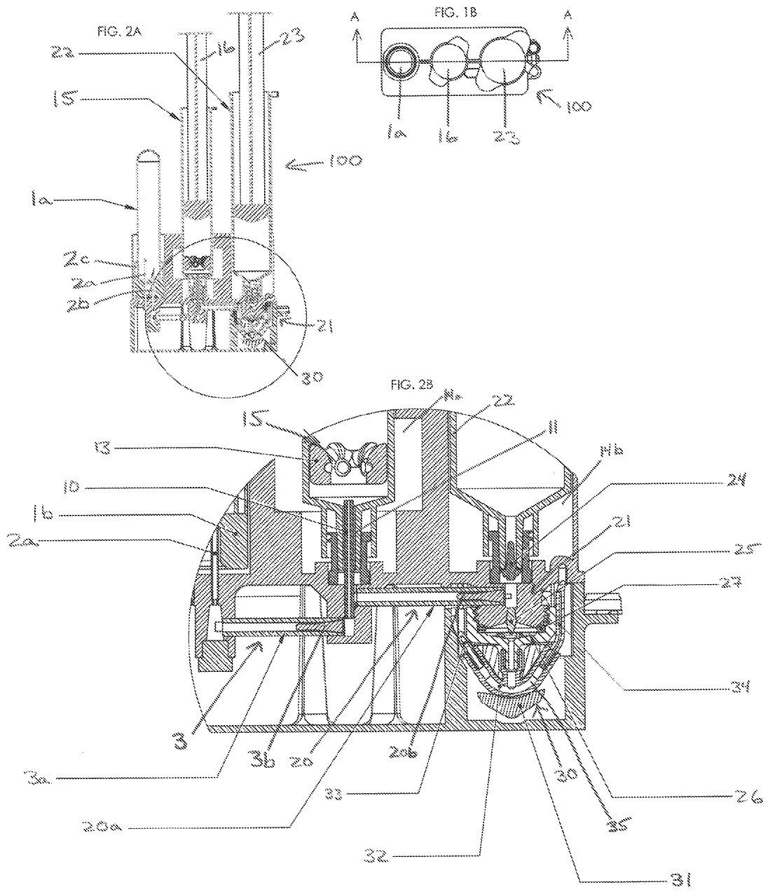

FIG. 2A shows a cross-sectional view of the exemplary automatic nucleic acid extraction cartridge (100) of FIG. 1B along line A-A. Shows a top view of the exemplary automatic nucleic acid extraction cartridge (100) of FIG. 1A. The cartridge or unit (100) comprises a sample port or sampling device (2) that receives a liquid biological sample in a liquid sample container (2c) and includes a sampling needle (2a), a venting needle (2b); a cell processing chamber or a cell lysis chamber (15) and first pressure exerting device (16); a wash fluid chamber (22) and a second pressure exerting device (23); and a filter assembly (21). The sample port (2) is in one-way fluid communication with the cell processing chamber (15), the cell processing chamber (15) is in one-way fluid communication with the filter assembly (21), the wash fluid chamber (22) is in fluid communication with the filter assembly (21), and the filter assembly (21) is in fluid communication with a diverter valve (30).

FIG. 2B shows the lower right portion of the exemplary automatic nucleic acid extraction cartridge (100) of FIG. 2A. The cartridge or unit (100) comprises a sampling needle (2a); a cell processing chamber or a cell lysis chamber (15); a wash fluid chamber (22); and a filter assembly (21) with a filter seal (27) for a filter member (26). The sampling needle (2a) is in one-way fluid communication with the cell processing chamber (15) via a first one-way valve (3) that includes a conduit (3a) that receives a partially conical or frustoconical elastomer valve pin (3b). The cell processing chamber (15) is in one-way fluid communication with the filter assembly (21) via a first one-way valve (20) that includes a conduit (20a) that receives a partially conical or frustoconical elastomer valve pin (20b). The wash fluid chamber (22) is in fluid communication with the filter assembly (21) via a third one-way valve (30) located downstream of a plug valve/plug (24) to assure materials from the filter assembly (21) does not enter the wash fluid chamber (22). The filter assembly (21) is in fluid communication with a diverter valve (30) with a pinching member or actuator (31) and (i) a waste conduit (33) when the diverter valve (30) is biased to the first reversibly sealable (or closable) output (32) and (ii) a pathogen nucleic acid conduit (34) when the diverter valve (30) is biased to the second reversibly sealable (or closable) output (35).

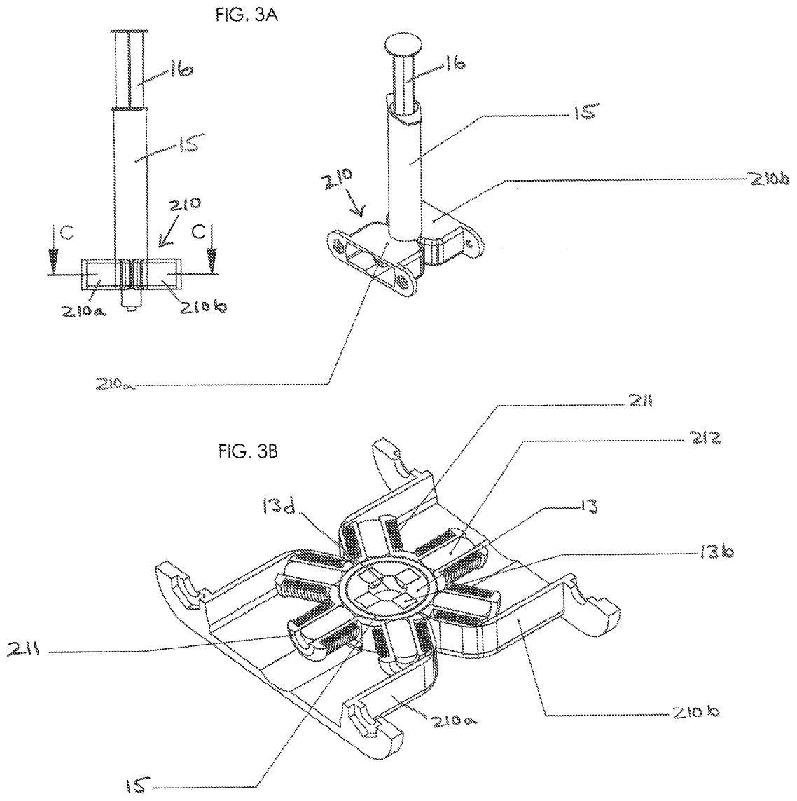

FIGS. 3A and 3B show an exemplary external magnetic field creating device (210) of the present disclosure. FIG. 3A shows an exemplary external magnetic field creating device (210) having two arms (210a, 210b) that are surrounding an exemplary cell processing chamber or cell lysing chamber (15) of the automatic nucleic acid extraction cartridge (100) of FIG. 1A. FIG. 3B shows a cross-sectional view of the exemplary external magnetic field creating device (210) surrounding the exemplary cell processing chamber (15) of FIG. 3A along line C-C, wherein the cell processing chamber (15) includes a mixing apparatus (13) having magnets (13b) and an open center (13d). The exemplary external magnetic field creating device (210) includes six field or stator coils (211), each wrapped around a magnetic core (212).

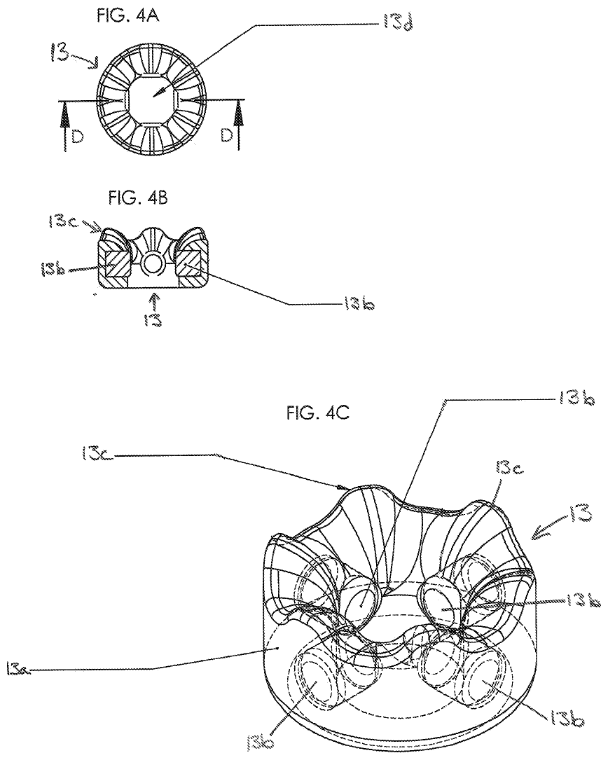

FIGS. 4A-4C show an exemplary mixing apparatus (13) of the present disclosure. FIG. 4A shows an exemplary mixing apparatus (13) of the present disclosure with an open center (3d). FIG. 4B shows a cross-sectional view of the exemplary mixing apparatus (13) of FIG. 4A along line D-D. The exemplary mixing apparatus includes a mixing body with (a) an open center, (b) magnets (13b) inserted in the mixing body, and (c) fluid engagement features that extend upward from the top of the mixing body (13a). FIG. 4C shows an exemplary mixing apparatus (13) of the present disclosure that includes a mixing body with (a) an open center, (b) magnets (13b) inserted in the mixing body, and (c) fluid engagement features that extend upward from the top of the mixing body (13a).

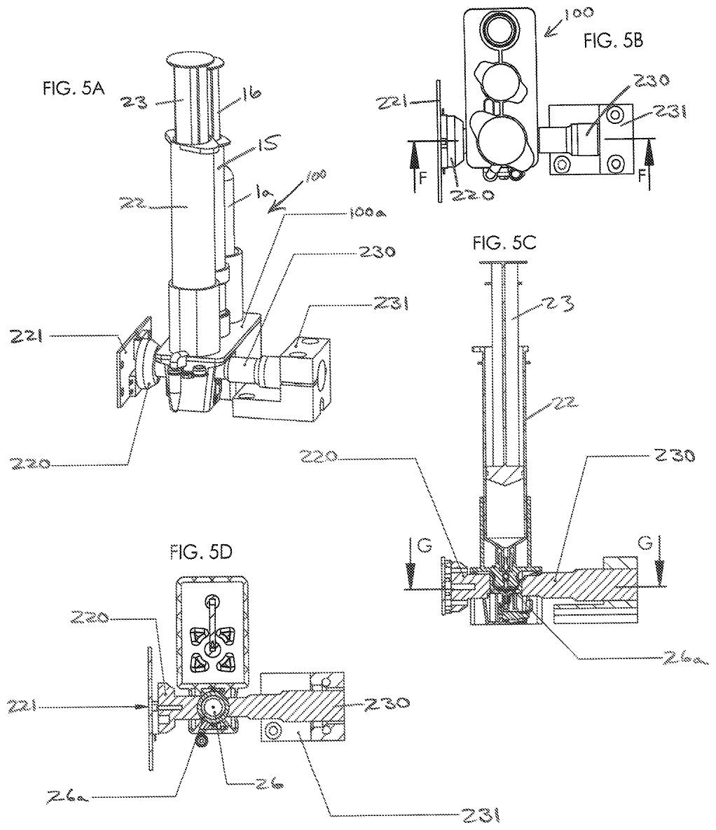

FIGS. 5A-5D shows an exemplary pathogen lysing device (220, 230) of the present disclosure engaged with the automatic nucleic acid extraction cartridge (100) of the present disclosure. FIG. 5A shows a side view of an exemplary heater (220) connected to a heater mount (221) and an exemplary sonic or ultrasonic wave transmitter (230) connected to an sonic or ultrasonic wave transmitter mount (231), the heater (220) and the wave transmitter (230) both engaged with an exemplary automatic nucleic acid extraction cartridge (100) of the present disclosure. FIG. 5B shows a top view of FIG. 5A. FIG. 5C shows a cross-sectional view of FIG. 5B along lines F-F. FIG. 5C shows a cross-sectional view of FIG. 5C along lines G-G.

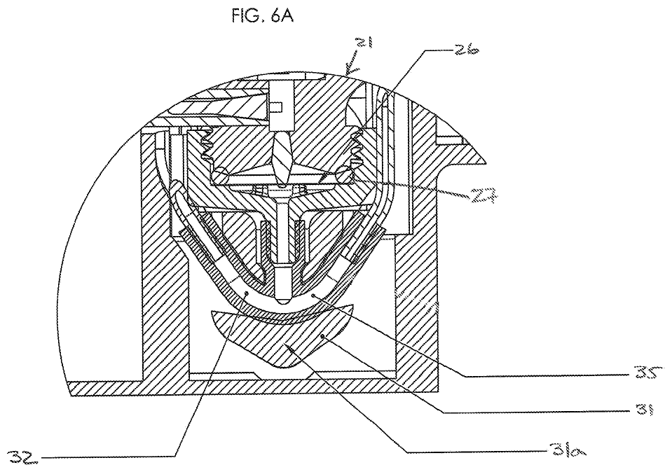

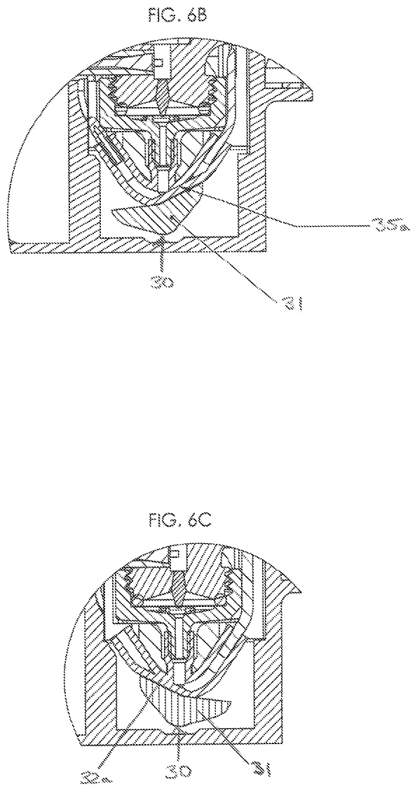

FIGS. 6A-6C show an exemplary diverter valve (30) of the present disclosure. FIG. 6A shows an exemplary diverter valve (30) having two outputs (32, 35) and a single actuator or pinching member (31) with a pivot point or point of rotation (31a) to facilitate the reversible pinching, compressing, or crimping of the outputs (32, 35). In this exemplary embodiment, the diverter valve is connected below the filter assembly (21). FIG. 6B shows the exemplary diverter valve (30) of FIG. 6A in which the actuator or pinching member (31) is rotated to reversibly sealed or close output 35 (35a). FIG. 6C shows the exemplary diverter valve (30) of FIG. 6A in which the actuator or pinching member (31) is rotated to reversibly sealed or close output 32 (35a).

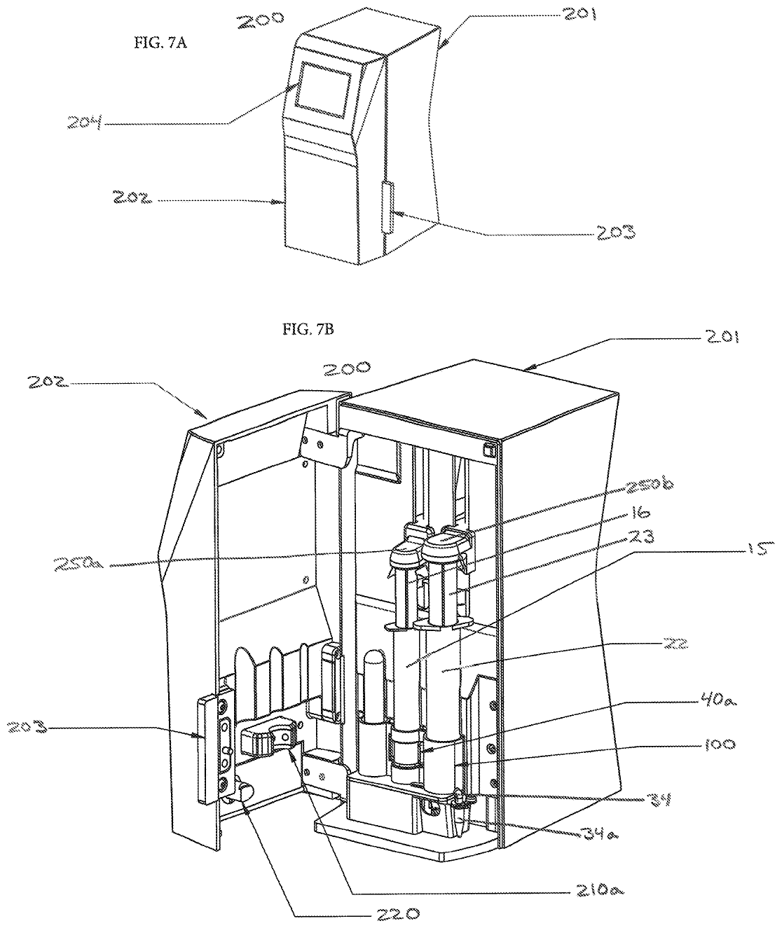

FIG. 7A shows an exemplary automated nucleic acid extraction system (200) of the present disclosure comprising a system body (201) and a system door (202) that is attached to and articulates with respect to the system body (201). In this view, the system door (202) is in a closed position that creates an internal chamber that the cartridge (100) of the present disclosure is located. The exemplary door comprises a latch (203) (for example, a magnetic release) and a display (204), which may include a touchscreen input. FIG. 7B show an exemplary automated nucleic acid extraction system (200) of the present disclosure in which the system door (202) that is attached to and articulates with respect to the system body (201) is open.

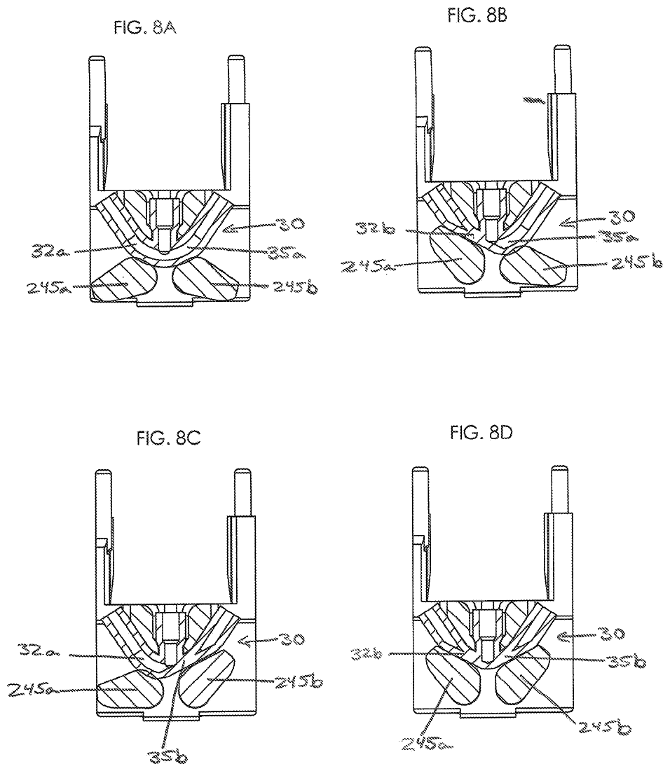

FIGS. 8A, 8B, 8C, and 8D show an exemplary diverter valve (30) having two actuators or pinching members (245a, 245b) that independently pinches, compresses, or crimps the reversibly sealable (or closable) output (32, 35), thereby resulting in a sealed (or closed) output (32a, 35a).

FIG. 9 shows an exemplary diverter control unit (240) to articulate an actuator or pinching member (31, 245a, 245b).

### REFERENCE NUMBERS

100—Automatic nucleic acid extraction cartridge
100a—Cartridge housing
1a—Liquid sample container
1b—Septum of liquid sample container
2—Sample port or sampling device
2a—Sampling needle
2b—Venting needle
2c—Liquid sample container holder
3—First one-way valve
3a—Conduit of first one-way valve
3b—Partially conical elastomer valve pin or a frustoconical elastomeric valve pin of first one-way valve
10—First conduit
11—Second conduit
13—Mixing apparatus
13a—Mixing body
13b—Magnet of the mixing body
13c—Fluid engagement features of the mixing body
13d—Open center of the mixing body
14a—First syringe holder
14b—Second syringe holder
15—Cell processing chamber or cell lysis chamber
16—First pressure exerting device
16a—Processing port
20—Second one-way valve
20a—Conduit of second one-way valve
20b—Partially conical elastomer valve pin or a frustoconical elastomeric valve pin of second one-way valve
21—Filter Assembly
22—Wash fluid chamber
23—Second pressure exerting device
23a—Washing fluid chamber port
24—Plug valve/plug
25—Third one-way valve
26—Filter member
26a—Filter housing
27—Filter seal
30—Diverter valve
31—Pinching member or actuator
32—First reversibly sealable (or closable) output
32a—Open first reversibly sealable (or closable) output
32b—Closed first reversibly sealable (or closable) output
33—Waste conduit
33a—Waste reservoir or tank
34—Pathogen nucleic acid conduit
34a—Extracted nucleic acid receptacle
35—Second reversibly sealable (or closable) output
35a—Open second reversibly sealable (or closable) output
35b—Closed second reversibly sealable (or closable) output
40a—A magnetic field window or access point
40b—A pathogen lysis window or access
200—Automated nucleic acid extraction system
201—System body
202—System door
203—Latch or magnetic release
204—Display and/or touchscreen input
210—External magnetic field (ExMF) creating device
210a—Arm of the external magnetic field (ExMF) creating device
210b—Arm of the eternal magnetic field (ExMF) creating device
211—Field or stator coil
212—Magnetic core of the field or stator coil
220—Pathogen lysing device, heater
221—Heater mount
230—Pathogen lysing device, sonic or ultrasonic wave transmitter
231—Sonic or ultrasonic wave transmitter mount
240—Diverter valve control unit
241a—First servo motor
241b—Second servo motor
242a—First servo motor arm
242b—Second servo motor arm
243a—First servo shaft
243b—Second servo shaft
244a—First rotating actuator shaft
244b—First rotating actuator shaft
245a—First actuator or pinching member
245b—First actuator or pinching member
250a—First driver
250b—Second Driver

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

Thus, those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. An automatic nucleic acid extraction cartridge comprising:
a housing comprising,
a sample port that receives a liquid biological sample;
a cell processing chamber;
a wash fluid chamber;
a filter assembly comprising a filter member; and
a diverter valve having a first reversibly sealable output and a second reversibly sealable output,
wherein
the sample port is in one-way fluid communication with the cell processing chamber,
the cell processing chamber is in one-way fluid communication with the filter assembly,
the wash fluid chamber is in fluid communication with the filter assembly, and
the filter assembly is in fluid communication with the diverter valve and (i) a waste conduit when the diverter valve is biased to the first reversibly sealable output and (ii) a pathogen nucleic acid conduit when the diverter valve is biased to the second reversibly sealable output.

2. The automatic nucleic acid extraction cartridge of claim 1, wherein:
(a) a liquid sample container comprises a liquid sample contained therein;
(b) the cell processing chamber comprises a processing solution contained therein;
(c) the cell processing chamber comprises a mixing apparatus contained therein;
(d) the wash fluid chamber comprises a wash solution contained therein;
(e) the filter member is configured to retain one or more pathogens;
(f) the one or more pathogens comprises one or more bacteria, one or more virus, one or more eukaryotic pathogen, or a combination thereof;
(g) the first reversibly sealable output includes a region that can be reversibly pinched, compressed, or crimped to be in a sealed position that does not allow fluid to pass through;
(h) the second reversibly sealable output includes a region that can be reversibly pinched, compressed, or crimped to be in a sealed position that does not allow fluid to pass through;
(i) the first reversibly sealable output and the second reversibly sealable output are both in a sealed position;
(j) the sample port and the cell processing chamber, the cell processing chamber and the filter assembly, the wash fluid chamber and the filter assembly, the filter member or filter assembly and the diverter valve are connected via a conduit;
(k) the filter member has a pore size that retains one or more pathogens;
(l) the filter member has a coating that has an affinity for or captures one or more pathogens;
(m) the filter member is statically charged;
(n) the filter member is made of a material that does not induce a fibrinogen-driven clotting reaction;
(o) the processing solution makes the non-pathogenic components of the liquid sample filterable without lysing one or more bacterial pathogens, one or more eukaryotic pathogens, one or more viral pathogens, or a combination thereof;
(p) the processing solution is a hypertonic solution relative to one or more non-pathogen eukaryotic cells of the liquid sample; or
(q) the processing solution (i) does not lyse or break down one or more prokaryotic pathogens, (ii) does not lyse or breakdown one or more eukaryotic pathogens, (iii) does not lyse or break down one or more viral pathogens, (iv) lyses blood cells, (v) breaks down proteins, (vi) breaks down nucleic acids, (vii) suppresses clotting, (viii) lyses non-pathogen eukaryotic cells, or (ix) a combination thereof; or
(r) a combination thereof.

3. The automatic nucleic acid extraction cartridge of claim 1, wherein
(i) the cartridge further comprises:
(a) a sampling device that is inserted into a liquid sample container
(b) a liquid sample container holder;
(c) a first pressure exerting device in fluid communication with the cell processing chamber;
(d) a second pressure exerting device in fluid communication with the wash fluid chamber;
(e) an extracted nucleic acid receptacle that is in fluid communication with the pathogen nucleic acid conduit;
(f) a waste reservoir that is in fluid communication with the waste conduit or the first reversibly sealable output; or
(g) a combination thereof;
(ii) the filter assembly further comprises:
(a) a thermally conductive element that transmits thermal energy to the filter member;
(b) a metal element that transmits at least vibrational energy to the filter member; or
(c) a combination thereof; or
(iii) a combination thereof.

4. The automatic nucleic acid extraction cartridge of claim 2, wherein:
(a) the conduit connecting the sample port and the cell processing chamber comprises a first one-way valve;
(b) the conduit of the cell processing chamber and the filter assembly comprises a second one-way valve;
(c) the conduit of the wash fluid chamber and the filter assembly comprises a third one-way valve;
(d) the mixing apparatus is a rotary mixer comprising a mixing body; or
(e) a combination thereof.

5. The automated nucleic acid extraction cartridge of claim 4, wherein the first one-way valve, the second one-way valve, the third one-way valve, or a combination thereof, comprise:
a conduit having a hollow interior that includes a straight interior region and an expanding conical interior region located at the end of the conduit that fluid exits the one-way valve, and
a partially conical elastomer valve pin or a frustoconical elastomeric valve pin that mates with the expanding conical interior region of the conduit, thereby causing a substantially or completely water-tight seal to form when there is flow or positive pressure from a fluid contacting the larger end of the valve pin.

6. The automated nucleic acid extraction cartridge of claim 5, wherein
(a) the valve pin comprises an extended cylindrical area that extends from the small end of the pin and that is smaller in diameter from the straight interior region of the one-way valve;
(b) the taper angle of the expanding conical interior region and the valve pin is about 12 degrees to about 24 degrees;
(c) the valve pin has a slightly greater taper angle than the expanding conical interior region of the valve; or
(d) a combination thereof.

7. The automated nucleic acid extraction cartridge of claim 3, wherein
(a) the sampling device is inserted into the liquid sample in an upward direction;
(b) the sampling device punctures the liquid sample container;
(c) the sampling device includes a sampling needle and a venting needle, each being inserted into the liquid sample in the liquid sample container;
(d) the first pressure exerting device is capable of providing positive pressure and negative pressure to the cell processing chamber;
(e) the first pressure exerting device moves fluid into the cell processing chamber when a negative pressure is applied by the first pressure exerting device to the cell processing chamber;
(f) the first pressure exerting device moves fluid into the filter assembly when a positive pressure is applied by the first pressure exerting device to the cell processing chamber;
(g) the second pressure exerting device is capable of providing at least positive pressure to the wash fluid chamber;
(h) the filter member retains one or more pathogens when the second pressure exerting device applies a positive pressure to the wash fluid chamber, thereby passing a portion of the wash solution through the filter assembly;
(i) the extracted nucleic acid receptable receives the extracted nucleic acids when the second pressure exerting device passes a portion of the wash solution through the filter assembly after the one or more pathogens are lysed from the second reversibly sealable output;
(j) the first pressure exerting device is a processing port that is capable of engaging a pressure driver from an external instrument that provides positive pressure and negative pressure to the processing port;
(k) the second pressure exerting device is a wash fluid chamber port that is capable of engaging a pressure driver from an external instrument that provides at least positive pressure to the wash fluid chamber port; or
(l) a combination thereof.

8. The automated nucleic acid extraction cartridge of claim 7, wherein:
(a) the sampling needle has
(i) a larger transfer capacity than the venting needle,
(ii) a shorter length than the venting needle, or
(iii) both;
(b) the venting needle vents to the waste reservoir and/or the atmosphere,
(c) the processing port seals the cell processing chamber;
(d) the wash fluid chamber port seals the wash fluid chamber from the external environment; or
(e) a combination thereof.

9. The automated nucleic acid extraction cartridge of claim 4, wherein
(a) the cell processing chamber includes a circular cross-section and the rotary mixer comprises a cylindrical mixing body that rotates freely within the circular cross-section of the cell processing chamber;
(b) the rotary mixer further comprises two or more magnets inserted into the mixing body; or
(c) a combination thereof.

10. The automated nucleic acid extraction cartridge of claim 9, wherein
(a) the two or more magnets are substantially evenly spaced or substantially symmetrically located about the rotational axis of the rotary mixer;
(b) the two or more magnets have the same orientation of their polarity;
(c) the mixing apparatus further comprises fluid engagement features extending upward from the cylindrical mixing body;
(d) the cylindrical mixing body further comprises an open center, or
(e) a combination thereof.

11. The automated nucleic acid extraction cartridge of claim 10, wherein (i) the fluid engagement features are smooth enough to minimize cavitation or substantially eliminate cavitation, (ii) the fluid engagement features have, or the top of the cylindrical mixing body has, a substantially sinusoidal shape, (iii) the fluid engagement features create a vortex when spinning about a vertical axis, or (iv) a combination thereof.

12. The automated nucleic acid extraction cartridge of claim 3, further comprising
(a) a first syringe holder that accepts a first syringe wherein the cell processing chamber is the hollow cylinder of the first syringe and the first pressure exerting device is a sliding plunger of the first syringe;
(b) a second syringe holder that accepts a second syringe, wherein the wash fluid chamber is a hollow cylinder of the second syringe and the second pressure exerting device is a sliding plunger of the second syringe; or
(c) a combination thereof.

13. An automated nucleic acid extraction system comprising the cartridge of claim 1 and a system body that comprises:
(a) a first driver that provides the force to the first pressure exerting device to exert the negative pressure to the cell processing chamber and the positive pressure to the cell processing chamber;
(b) a second driver that provides the force to the second pressure exerting device to exert a positive pressure to the wash fluid chamber;
(c) an external magnetic field (ExMF) creating device that creates an ExMF that drives the mixing apparatus;
(d) a pathogen lysing device that engages the filter assembly and that lyses the one or more pathogens contained in the filter assembly; and
(e) a diverter valve control unit that controls the diverter valve.

14. The automated nucleic acid extraction system of claim 13, wherein
(a) the system body further comprises a control unit that controls the first driver, the second driver, the ExMF creating device, the pathogen lysing device, the diverter valve, or a combination thereof;
(b) the system further comprises a system door that is attached to and articulated with the system body between an open position in which the cartridge is accessible and a closed position that produces an enclosed space where the cartridge is placed or located;
(c) the ExMF creating device includes two arms that together surround the cell processing chamber for creating the ExMF, each arm including one or more substantially evenly spaced or substantially symmetrically located field or stator coils;
(d) the ExMF creating device senses the position of the one or more magnets of the rotary mixer relative to the field or stator coil using the back electromagnetic field (EMF) created by the permanent magnets of the rotary mixer passing by the field or stator coils;
(e) the ExMF creating device continuously examines whether the rotary mixer is rotating;
(f) the ExMF creating device provides continuous intimation of how the rotary mixer is rotating;
(g) the mixing apparatus (i) mixes at about 500 to about 7000 rotations per minute (RPM),
(ii) mixes at least while the liquid sample is introduced into the cell processing chamber,
(iii) mixes the liquid sample and the processing solution for about 3 to about 10 minutes,
or (iv) a combination thereof;
(h) the diverter valve control unit comprises a first actuator or pinching member for the first reversibly sealable output and a second actuator or pinching member for the second reversibly sealable output, wherein each actuator has a first position that seals the output and a second position that opens the output;
(i) the first driver further comprises at least one force sensor that detects how much force is being exerted on the cell processing chamber,
(j) the second driver further comprises at least one force sensor that detects how much force is being exerted on the wash fluid chamber,
(k) the system body further comprising a waste reservoir that is in fluid communication with the end of the waste conduit not connected to the diverter valve;
(l) the mixing apparatus is a rotary mixer, and the ExMF creating device includes (i) two or more field or stator coils that are sequentially energized and that are placed around the cell processing chamber and in the same field as the rotatory mixer, or (ii) two or more synchronized magnets that are rotated around the cell processing chamber and in the same plane as the rotary mixer; or
(m) a combination thereof.

15. The automated nucleic acid extraction system of claim 14, wherein
(a) the two arms are articulated between a closed position that places the two arms around the cell processing chamber for creating the ExMF and an open position that permits the cell processing chamber to be positioned between the two arms;
(b) one of the two arms of the ExMF creating device is mounted on the system body and the second of the two arms of the ExMF creating device is mounted on the system door; or
(c) the first position of each of the actuators closes the output by pinching, compressing, or crimping a region of the output that can be reversibly pinched, compressed, or crimped.

16. The automated nucleic acid extraction system of claim 13, wherein
(a) the diverter valve control unit comprises an actuator or pinching member that has
a first position that pinches, compresses, or crimps for the first reversibly sealable output,
a second position that pinches, compresses, or crimps for the second reversibly sealable output, and
a third position that does not pinch, compress, or crimp either of the first or the second reversibly sealable outputs to the point of stopping the flow of fluid;
(b) the pathogen lysing device comprises
a heater that heats contents of the filter assembly to a temperature sufficient to lyse the one or more pathogens;
a sonic or ultrasonic wave transmitter that transmits sound waves to contents of the filter assembly that are sufficient to lyse the one or more pathogens; or
a combination thereof; or
(c) a combination thereof.

17. A mixing apparatus that comprises a cylindrical mixing body that comprises two or more magnets inserted into the cylindrical mixing body.

18. The mixing apparatus of claim 17, wherein
(i) the two or more magnets have the same orientation of their polarity;
(ii) the mixing apparatus further comprises fluid engagement features extending upward from the cylindrical mixing body;
(iii) the cylindrical mixing body further comprises an open center;
(iv) the mixing apparatus further comprising an external magnetic field (ExMF) creating device that creates an ExMF that drives the mixing apparatus;
(v) the mixing apparatus mixes at about 500 to about 7000 rotations per minute (RPM); or
(vi) a combination thereof.

19. The mixing apparatus of claim 18, wherein (i) the fluid engagement features are smooth enough to minimize cavitation or substantially eliminate cavitation, (ii) the fluid engagement features have, or the top of the cylindrical mixing body has, a substantially sinusoidal shape, (iii) the fluid engagement features create a vortex when spinning about a vertical axis, or (iv) a combination thereof.

20. The mixing apparatus of claim 18, wherein
(a) the mixing apparatus or the cylindrical mixing body is a rotary mixer, and the ExMF creating device includes (i) two or more field or stator coils that are sequentially energized and that are placed around the cell processing chamber and in the same field as the rotatory mixer, or (ii) two or more synchronized magnets that are rotated around the cell processing chamber and in the same plane as the rotary mixer;
(b) the ExMF creating device includes two arms that together surround the cylindrical mixing body for creating the ExMF, each arm including one or more substantially evenly spaced or substantially symmetrically located field or stator coils;
(c) the ExMF creating device senses the position of the one or more magnets of the cylindrical mixing body or the rotary mixer relative to the field or stator coil using the back electromagnetic field (EMF) created by the permanent magnets of the rotary mixer passing by the field or stator coils;
(d) the ExMF creating device continuously examines whether the cylindrical mixing body or the rotary mixer is rotating;
(e) provides continuous intimation of how the cylindrical mixing body or the rotary mixer is rotating; or
(f) a combination thereof.

21. The mixing apparatus of claim 20, wherein
(a) the two arms are articulated between a closed position that places the two arms around the cylindrical mixing body for creating the ExMF and an open position that permits the cylindrical mixing body to be positioned between the two arms; or
(b) one of the two arms of the ExMF creating device is mounted on a first body and the second of the two arms of the ExMF creating device is mounted on a second body, wherein the first body and second body are brought together such that the two arms surround the cylindrical mixing body for creating the ExMF.

22. A diverter valve comprising
an input that splits at a single location into at least two outputs, each output including a conduit, or a region thereof, that can be reversibly pinched, compressed, or crimped, thereby stopping the flow of fluid through the output when pinched, compressed, or crimped; and
at least one actuator or pinching member that pinches the at least two outputs.

23. The diverter valve of claim 22, wherein:
(a) the at least two outputs is a first output and a second output, and the at least one actuator or pinching member is a single actuator or pinching member that has (i) a first position that pinches the first output and does not pinch the second output, (ii) a second position that does not pinch the first output or the second output, and (iii) a third position that pinches the second output and does not pinch the first output;
(b) there is an actuator or pinching member for each output;
(c) the at least one actuator or pinching member is located just after the split resulting in zero dead volume; or
(d) the conduit or region thereof is comprised of an elastomeric material that can be reversibly pinched, compressed, or crimped, thereby stopping the flow of fluid through the output when pinched, compressed, or crimped; or
(e) a combination thereof.

24. The diverter valve of claim 23, wherein the single actuator or pinching member is an elongated actuator or pinching member whose center point of its length is substantially located and pressed against the split without disrupting the flow when in the second position, and the elongated actuator or pinching member rotates between (i) the first position to direct the flow to the second output and (ii) the third position to direct the flow to the first output, wherein the second position is between the first position and the second position.

25. A method of performing nucleic acid extraction, the method comprising:
providing a automated nucleic extraction cartridge claim 1 or a system comprising the automated nucleic acid extraction cartridge;
transferring a liquid sample from the liquid sample container into the cell processing chamber;
mixing the liquid sample with a processing solution in the cell processing chamber;
transferring the processed liquid sample from the cell processing chamber to the filter assembly;
washing the processed liquid sample through the filter member with a wash solution, wherein the one or more pathogens are retained on the filter member and the filtrate is directed to the waste conduit by way of the diverter valve;
lysing the one or more pathogens through non-chemical means; and
isolating extracted nucleic acids from the one or more pathogens with wash solution, wherein the diverter valve directs the filtrate to the pathogen nucleic acid conduit,
optionally:
the method further comprises inserting the sampling device into the liquid sample container comprising a liquid sample;
the method is automated; or
a combination thereof.
